(19) United States
(12) Patent Application Publication
(10) Pub. No.: US 2019/0134004 A1
(43) Pub. Date: May 9, 2019

(54) METHODS AND COMPOSITIONS FOR TREATING BREAST AND PROSTATE CANCER

(71) Applicant: The University of Chicago, Chicago, IL (US)

(72) Inventors: Suzanne D. Conzen, Chicago, IL (US); Russell Szmulewitz, Chicago, IL (US)

(21) Appl. No.: 16/306,626

(22) PCT Filed: Jun. 16, 2017
(86) PCT No.: PCT/IB2017/053601

§ 371 Date: Dec. 3, 2018

Related U.S. Application Data
(60) Provisional application No. 62/351,023, filed on Jun. 16, 2016.
(60) Provisional application No. 62/383,799, filed on Sep. 6, 2016.

(51) Int. Cl. A61K 31/4166; A61K 31/337; A61K 31/137; A61K 31/167; A61K 31/58; A61K 31/573; A61K 31/496; A61K 45/06; A61P 35/04

(52) U.S. Cl. CPC A61K 31/4166; A61K 31/337; A61K 31/137; A61K 31/167; A61P 35/04; A61K 31/573; A61K 31/496; A61K 45/06; A61K 31/58

(57) ABSTRACT

The current disclosure relates to combination treatments for breast cancers such as TNBC and for prostate cancers. Embodiments concern methods, compositions, and apparatuses for treating breast cancer and prostate cancer patients. Aspects relate to a method of inhibiting proliferation of glucocorticoid receptor positive (GR+) breast or prostate cancer cells comprising administering to the cells an effective amount of a BET inhibitor in combination with one or both of a chemotherapeutic agent and a glucocorticoid receptor modulator.

### CROSS-REFERENCE TO RELATED APPLICATIONS

This application claims priority to U.S. Provisional Patent Application Ser. No. 62/351,023 filed Jun. 16, 2016, and U.S. Provisional Patent Application Ser. No. 62/383,799 filed Sep. 6, 2016. The entire contents of each of the above-referenced disclosures are specifically incorporated herein by reference without disclaimer.

### BACKGROUND OF THE INVENTION

### I. Field of the Invention

Embodiments of this invention are directed generally to biology and medicine. In certain aspects methods involve treating a breast or prostate cancer patient.

### II. Background

There are over 1 million cases of breast cancer per year on a global basis, of which around 0.5 million are in the US, 40,000 are in the UK and nearly 2,000 in Ireland. It is the leading cause of cancer deaths among women (Keen and Davidson, 2003). Although the overall incidence of the disease is increasing within the western world, wider screening and improved treatments have led to a gradual decline in the fatality rate of about 1% per year since 1991. Inheritance of susceptibility genes, such as BRCA1 and BRCA2, account for only 5% of breast cancer cases and the factors responsible for the other 95% remain obscure (Grover and Martin, 2002). In the absence of a strategy to reduce causative agents of breast cancer, early detection remains the best approach to reducing the mortality rate of this disease. It is widely held that breast cancer initiates as the pre-malignant stage of atypical ductal hyperplasia (ADH), progresses into the pre-invasive stage of ductal carcinoma in situ (DCIS), and culminates in the potentially lethal stage of invasive ductal carcinoma (IDC). This linear model of breast cancer progression has been the rationale for the use of detection methods such as mammography in the hope of diagnosing and treating breast cancer at earlier clinical stages (Ma et al., 2003).

As more molecular information is being collated, diseases such as breast cancer are being sub-divided according to genetic signatures linked to patient outcome, providing valuable information for the clinician. Emerging novel technologies in molecular medicine have already demonstrated their power in discriminating between disease sub-types that are not recognizable by traditional pathological criteria (Sorlie et al., 2001) and in identifying specific genetic events involved in cancer progression (Srinivas et al., 2002).

One sub-type of breast cancer is triple negative breast cancer (TNBC). In TNBC, the offending tumor is estrogen receptor-negative, progesterone receptor-negative and HER2-negative. Because of its triple negative status, however, triple negative tumors generally do not respond to receptor targeted treatments. Depending on the stage of its diagnosis, triple negative breast cancer can be particularly aggressive, and more likely to recur than other subtypes of breast cancer. Few targetable molecular drivers have been identified for TNBC and thus standard treatment is limited to non-selective chemotherapy. Therefore, there is a need in the art for more effective therapies for breast cancer and specifically for TNBC.

Despite recent advances, the challenge of cancer treatment, including breast cancer therapy remains. Progress is limited with respect to the development of specific treatment regimens to clinically distinct tumor types, and to personalize tumor treatment in order to maximize outcome and efficiency. Therefore, there is a need in the art for therapeutic regimens that are based on a patient's breast cancer type.

### SUMMARY OF THE INVENTION

The current disclosure relates to combination treatments for breast cancers such as TNBC and for prostate cancers. Embodiments concern methods, compositions, and apparatuses for treating breast cancer and prostate cancer patients. Aspects relate to a method of inhibiting proliferation of androgen receptor positive (AR+) and/or glucocorticoid receptor positive (GR+) breast or prostate cancer cells comprising administering to the cells an effective amount of a BET (Bromodomain and Extraterminal Domain) inhibitor in combination with one or both of a chemotherapeutic agent and a glucocorticoid receptor modulator.

Further aspects relate to a method for treating a GR+ and/or AR+ breast or prostate cancer in a patient comprising administering an effective amount of a BET inhibitor in combination with one or both of a chemotherapeutic agent and a glucocorticoid receptor modulator.

Yet further aspects of the disclosure relate to a method of inhibiting proliferation of prostate cancer cells comprising administering to the cells an effective amount of a BET inhibitor in combination with one or both of an anti-androgen and a glucocorticoid receptor modulator.

Further aspects relate to a method for treating a prostate cancer and/or breast cancer in a patient comprising administering an effective amount of a BET inhibitor in combination with one or both of an anti-androgen and a glucocorticoid receptor modulator.

Further aspects relate to a method of inhibiting proliferation of glucocorticoid receptor positive (GR+) and/or androgen receptor (AR+) breast or prostate cancer cells comprising administering to the cells an effective amount of a BET inhibitor in combination with one or both of a chemotherapeutic agent and an anti-androgen.

In some embodiments, the cells or cancer are GR+. In some embodiments, the patient has previously been treated with one or more anti-androgens or one or more chemotherapeutic agents. In some embodiments, the patient has been determined to be chemo-resistant, resistant to the anti-androgen, or have a reduced sensitivity to a chemotherapeutic agent or an anti-androgen. In some embodiments, an anti-androgen comprises androgen deprivation therapy. Examples of androgen deprivation therapy comprises chemical castration methods such as LHRH (lutenizing hormone-releasing hormone) agonists or antagonists such as leuprolide, goserelin, triptorelin, histrelin, and degarelix.

In some embodiments, the cells or cancer are breast cancer. In some embodiments, the breast cancer is triple negative breast cancer (TNBC).

In some embodiments, the cells are prostate cancer cells or the cancer is prostate cancer. In some embodiments, the prostate cancer is castration resistant prostate cancer. In related embodiments, the method comprises administration of a BET inhibitor and one or more chemotherapeutic agent. In some embodiments, the methods further comprise administration of a chemotherapeutic agent. In some embodiments, the chemotherapeutic agent is one described herein or known in the art. In some embodinements, the chemotherapeutic agent comprises one or more of docetaxel, cabazitaxel, mitoxantrone, abiraterone, prednisone, radium-223, sipuleucel-T, mitoxantrone, bicalutamide, flutamide, nilutamide, ketoconazole, and low-dose corticosteroids.

In some embodiments, the cells or cancer are resistant to a chemotherapeutic. In some embodiments, the cells or cancer are resistant to antiadrogens. In some embodiments, the cells or cancer are resistant to enzalutamide. In some embodiments, the patient has been determined to have enzalutamide-resistant prostate cancer or a prostate cancer resistant to antiandrogens. In some embodiments, the patient is one that has been treated previously for prostate cancer with enzalutamide.

In some embodiments, the patient has previously been treated for breast or prostate cancer. In some embodiments, the patient has previously been treated with a chemotherapeutic agents. In some embodiments, the patient has been determined to be chemo-resistant or have a reduced sensitivity to a chemotherapeutic agent. In some embodiments, the cells or cancer are AR+. In some embodiments, the patient is determined to have cancer cells that are AR+. In some embodiments, the patient is determined to have cancer cells that are GR+. In some embodiments, the patient is determined to have cancer cells that are PR negative, ER negative, and HER-2 negative. In some embodiments, the patient is one that has been diagnosed as having GR+ cancer. In some embodiments, the patient is one that has been diagnosed as having TNBC.

In some embodiments, the methods further comprise administration of an AR modulator. In some embodiments, the AR modulator comprises Enobosarm (Ostarine, MK-2866, GTx-024), BMS-564,929, LGD-4033—(Ligandrol), AC-262,356, JNJ-28330835, LGD-2226, LGD-3303, S-40503, S-23, and RAD140.

In some embodiments, the methods comprise or further comprise administration of an antiandrogen. In some embodiments, the antiandrogen comprises one or more of chlormadinone acetate, cyproterone acetate, megestrol acetate, dienogest, drospirenone, oxendolone, spironolactone, bicalutamide, flutamide, nilutamide, apalutamide, darolutamide, enzalutamide, cimetidine, abiraterone acetate, VT-464, apalutamide, ODM-201, geleterone, topilutamide, and combinations thereof. In some embodiments, the antiandrogen comprises enzalutamide.

In some embodiments, the BET inhibitor and the glucocorticoid receptor modulator and/or chemotherapeutic agent are administered within one week of each other. In some embodiments, the BET inhibitor and the antiandrogen are administered within one week of each other. In some embodiments, the combination of anti-cancer compounds is administered within 24 hours of each anti-cancer compound. In some embodiments, the combination is administerd within 1, 6, 12, 24, 48 hours or 3, 4, 5, 6, 7, 8, 9, 10, 20, or 30 days (or any derivable range therein) of each anti-cancer compound. In some embodiments, the BET inhibitor is administered prior to or after the GR modulator, antiandrogen, and/or chemotherapeutic agent. In some embodiments, the BET inhibitor is administered for 1, 2, 3, 4, 5, 6, 7 days or 1, 2, 3, 4, 5, 6, 7, 8, 9, 10 weeks (or any derivable range therein) prior to administration of the GR modulator, antiandrogen, or chemotherapeutic. In some embodiments, the BET inhibitor and/or the GR modulator are administered for 1, 2, 3, 4, 5, 6, 7 days or 1, 2, 3, 4, 5, 6, 7, 8, 9, 10 weeks (or any derivable range therein) prior to administration of the chemotherapeutic agent and/or antiandrogen.

In some embodiments, the method comprises the administration of a GR modulator known in the art or described herein.

In some embodiments, the GR modulator is a drug or immunological agent that alters the ability of GR to function directly as a transcription factor and/or function as a GR chromatin modulator, thereby indirectly affecting gene expression in a specifically GR− dependent manner.

GR is a critical target of BET inhibitors, since bromodomain proteins are required for GR activity and therefore, BET inhibitors are proposed to decrease GR function and as a cancer therapeutic in GR-overexpressing, chemotherapy-resistant cancers. GR modulators useful in the compositions and methods described herein include steroidal GR ligands binding and displacing GR agonists from the ligand binding domain (LBD), non-steroidal GR ligands binding and displacing GR agonists from the ligand binding doman (LBD), molecules that inactivate the Hsp (e.g. 70 and 90) family, interacting proteins with GR molecules that prevent GR-coactivators from interacting with GR molecules that prevent GR dimerization-thereby affecting transcription factor activity and GR-mediated chromatin organization, and molecules that interfere with BRG1, the central ATPase of the human SWI/SNF complex, which is critical for GR function. Examples of GR modulators include: Steroidal GR modulators (e.g. RU-486, RU-43044, CP-409069 ORG 214007, ZK-216348) include 11-Monoaryl and 11,21 Bisaryl steroids, 11Beta-Aryl conjugates of mifepristone, and non-steroidal modulators, including octahydrophenanthrenes, spirocyclic dihydropyridines, triphenyl methanes (e.g. AL082D06), chromens, dibenzyl analines, dihydroqinolones, pyrimidine diones, fused azedecalins (e.g. 113176 and CORT 108297), and indole sulfonamides.

BET inhibitors are a class of drugs with anti-cancer, immunosuppressive, and other effects in clinical trials. These molecules are inhibitors of Bromodomain and Extra-Terminal motif (BET) proteins such as BRD2, BRD3, BRD4, and BRDT. These inhibitors may prevent protein-protein interaction between BET proteins and acetylated histones and transcription factors. Examples of BET inhibitors include: JQ1, I-BET 151 (GSK1210151A), I-BET 762 (GSK525762), OTX-015, TEN-010 (Tensha therapeutics), CPI-203, RVX-208 (Resverlogix Corp), LY294002, MK-8628 (Merck/Mitsubishi Tanabe), BMS-986158 (Bristol-Myers Squibb), INCB54329 (Incyte Pharmaceuticals), ABBV-075 (AbbVie, also called ABV-075), CPI-0610 (Constellation Pharmaceuticals/Roche), FT-1101 (Forma Therapeutics/Celgene), GS-5829 (Gilead Sciences), and PLX51107 (Daiichi Sankyo).

In some embodiments, the method comprises the administration of one or more chemotherapeutic agents. In some embodiments, the chemotherapeutic agent comprises one or more of capecitabine, carboplatin, cyclophosphamide, daunorubicin, docetaxel, doxorubicin, epirubicin, fluorouracil, gemcitabine, eribulin, ixabepilone, methotrexate, mitomycin C, mitoxantrone, paclitaxel, thiotepa, vincristine, or vinorelbine. In some embodments, the chemotherapeutic agent comprises one or more chemotherapeutic agents described herein.

In some embodiments, the method further comprises categorizing the patient as ER+ or ER− based the level of estrogen receptor expression and a predetermined threshold value for ER expression. In some embodiments, the method further comprises categorizing the patient as GR+ or GR− based the level of glucocorticoid receptor expression and a predetermined threshold value for GR expression. In some embodiments, the method further comprises categorizing the patient as PR+ or PR− based the level of progesterone expression and a predetermined threshold value for PR expression. In some embodiments, the method further comprises categorizing the patient as HER-2+ or HER-2-negative-based the level of HER-2 expression and a predetermined threshold value for HER-2 expression. In some embodiments, the method further comprises categorizing the patient as AR+ or AR-negative based on the level of AR expression and a predetermined threshold value for AR expression. In some embodiments, the predetermined threshold value identifies a patient as positive if the patient's expression level is in the 25^{th} percentile or greater compared to a normalized sample. In some embodiments, the normalized sample is based on one or more cancer samples. In some embodiments, the predetermined threshold value for GR activity is dependent on whether the patient is categorized as ER+ or ER. In some embodiments, the predetermined threshold value for GR activity identifies a patient as GR+ if the patient is ER- and GR activity level is in the 65^{th} percentile or greater compared to a normalized sample.

In some embodiments, the normalized sample is based on one or more cancer samples. In some embodiments, the method further comprises determining the activity or expression level of GR in a biological sample from the patient. In some embodiments, the activity level of GR is assayed by measuring the level of GR expression. In some embodiments, GR expression is GR transcript expression. In some embodiments, GR expression is GR protein expression. In some embodiments, the activity level of GR is measured by assaying the expression level of one or more GR-responsive genes. In some embodiments, the GR responsive gene is MCL1, SAP30, DUSP1, SGK1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, IDH2, SERPINF1, ERBB2, PECAM1, LBH, ST3GAL5, IL1R1, BIN1, WIPF1, TFPI, FN1, FAM134A, NRIP1, RAC2, SPP1, PHF15, BTN3A2, SESN1, MAP3K5, DPYSL2, SEMA4D, STOM, MAOA, AKAP1, AREG, ARHGEF26, BIRC3, CA12, CALCR, CDC42EP3, CYP24A1, DEPTOR, DOCK4, DUSP6, FGF18, FOS, GAD1, GREB1, IL6R, IL6ST, KAZN, KCNJ8, KDM4B, KIAA0226L, KLF9, LAMA3, MAFB, MYC, NR5A2, PERI, PHLDA1, PSCA, RGS2, RHOBTB1, SGK1, SNAI2, SOCS2, SYBU, TBC1D8, TGFA, WIPF1, WWC1, ALDH1A3, CXCL12, LRRC15, LY6H, NR4A2, PDZKl, PPIF. SLC22A4, RNF43, ARL14, CD44, CYP1A1, DDX10, EGR3, EMP1, FJX1, HCK, HEG1, HEY2, PTGES, RAB31, RARA, SIM1, SLC26A2, TMEM120B, TNFRSF11B, TRPC6, DIRAS2, KRT13, LRP4, PTGER4, RET, RGCC, SEMA3B, SERPINB9, SLC47A1, SUV39H2, RAPGEFL1, MICB, HS3ST3A1, HSPB8, IGFBP4, JAK2, KIT, LEF1, LINC00341, MAFF MYBL1, NPY1R, NPYSR, PGR, PLAC1, or PMAIP1.

Further aspects relate to a method for treating a triple-negative breast cancer patient determined to be GR+ comprising administering a BET inhibitor and administering a chemotherapeutic agent and/or a glucocorticoid receptor (GR) modulator. In some embodiments, the patient was previously determined to be chemotherapy-resistant.

Embodiments also cover apparatuses, kits, and computer readable medium and systems for assessing the level or activity of ER, GR, PR, HER-2 and/or other genes in a patient's breast or prostate cancer sample and determining a prognosis; and/or treating the patient accordingly. It is specifically contemplated that a breast cancer or prostate cancer patient is a human. Accordingly, in human patients, ER refers to an estrogen receptor in a human, GR refers to a glucocorticoid receptor in a human, and PR refers to a progesterone receptor in a human.

Methods include directly measuring or assaying the level of expression or activity refers to measuring or assaying a sample to determine the level of GR expression (protein or transcript) in the cell. Indirectly obtaining the level of expression includes measuring or assaying expression or activity of a gene or protein that correlates with GR expression or activity. In some embodiments, the level of GR and/or PR expression can be indirectly obtained by measuring or assaying expression of a GR or PR-responsive gene, which refers to a gene whose expression is affected in a dose-dependent manner by GR or PR expression or activity. Expression refers to either protein expression or RNA (transcript) expression. Methods may involve either type of expression and a variety of assays are well known to those of skill in the art. For example, quantitative PCR may be performed to obtain RNA expression levels. Alternatively, reagents to detect protein expression levels may be employed in embodiments. Methods may involve probes, primers, and/or antibodies that are specific to GR or ER in order to assess expression levels.

In some embodiments, the activity level of GR is measured by assaying the level of GR expression. In additional embodiments, GR expression is GR transcript expression. In other embodiments, GR expression is GR protein expression. As discussed above, in some embodiments, the activity level of GR is measured by assaying the expression level of one or more GR-responsive genes. A GR-responsive gene may be one or more of the following: MCL1, SAP30, DUSP1, SGK1, SMARCA2, PTGDS, TNFRSF9, SFN, LAPTMS, GPSM2, SORT1, DPT, NRP1, ACSLS, BIRC3, NNMT, IGFBP6, PLXNC1, SLC46A3, C14orf139, PIAS1, IDH2, SERPINF 1, ERBB2, PECAM1, LBH, ST3GAL5, IL1R1, BIN1, WIPF1, TFPI, FN1, FAM134A, NRIP1, RAC2, SPP1, PHF15, BTN3A2, SESN1, MAP3K5, DPYSL2, SEMA4D, STOM, MAOA, AKAP1, AREG, ARHGEF26, BIRC3, CA12, CALCR, CDC42EP3, CYP24A1, DEPTOR, DOCK4, DUSP6, FGF18, FOS, GAD1, GREB1, IL6R, IL6ST, KAZN, KCNJ8, KDM4B, KIAA0226L, KLF9, LAMA3, MAFB, MYC, NR5A2, PERI, PHLDAL PSCA, RGS2, RHOBTB1, SGK1, SNAI2, SOCS2, SYBU, TB C1D8, TGF A, WIPF1, WWC1, ALDH1A3, CXCL12, LRRC15, LY6H, NR4A2, PDZK1, PPIF. SLC22A4, RNF43, ARL14, CD44, CYP1A1, DDX10, EGR3, EMP1, FJX1, HCK, HEG1, HEY2, PTGES, RAB31, RARA, SIM1, SLC26A2, TMEM120B, TNFRSF11B, TRPC6, DIRAS2, KRT13, LRP4, PTGER4, RET, RGCC, SEMA3B, SERPINB9, SLC47A1, SUV39H2, RAPGEFL1, MICB, HS3ST3A1, HSPB8, IGFBP4, JAK2, KIT, LEF1, LINC00341, MAFF MYBL1, NPY1R, NPYSR, PGR, PLAC1, or PMAIP1.

The term “ER+” refers to a classification of ER expression that indicates the patient expresses estrogen receptor in cancer cells at or above a certain level. The term “ER-” refers to a classification of ER expression that indicates the patient expresses estrogen receptor at a relatively low level in cancer cells, meaning at or below a certain level. In embodiments of the invention, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein.

Methods may involve measuring the activity level of glucocorticoid receptor in a biological sample from the patient containing breast or prostate cancer cells and measuring the expression level of estrogen receptor in the biological sample.

In certain embodiments, the predetermined threshold value for ER expression identifies a patient as ER+ if the patient's ER expression level is in the 25^{th} percentile or greater compared to a normalized sample. This means the patient may be designated as having a level of ER expression that is at or above 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. It is contemplated that in some cases, a patient may be designated as ER+ if the patient's ER expression level is at or above 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. The patient may also be referred to as having a normal or high ER expression level. The higher the percentile, the higher the relative expression level.

In embodiments, methods may also involve categorizing the patient as GR+ r GR− based on a predetermined threshold value for GR activity. In some cases, a predetermined threshold value for GR activity is dependent on whether the patient is categorized as ER+ or ER−. Embodiments may involve a predetermined threshold value for GR activity that identifies a patient as GR+ if the patient is ER− and GR activity level is in the 65^{th} percentile or greater compared to a normalized sample. It is contemplated that in some cases, a patient may be designated as GR+ if the patient's GR expression level is at or above 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. The threshold value may or may not be dependent on GR expression levels or status. In some embodiments, the threshold value depends on whether the patient is GR− or not. The higher the percentile, the higher the relative expression level.

In certain embodiments, the predetermined threshold value for PR expression identifies a patient as PR− if the patient's PR expression level is in the 25^{th} percentile or lower compared to a normalized sample. Alternatively, the patient may be determined to be PR+ if above this percentile as compared to a reference sample. This means the patient may be designated as having a level of PR expression that is at or above 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. It is contemplated that in some cases, a patient may be designated as PR+ if the patient's PR expression level is at or above 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. The patient may also be referred to as having a normal or high PR expression level. The higher the percentile, the higher the relative expression level. It is further contemplated that in some cases, a patient may be designated as PR− if the patient's PR expression level is at or below 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 percentile, or any range derivable therein. In some embodiments, the patient's cancer is designated at PR-when the expression of PR is at or below the 20^{th} or 10^{th} percentile. The patient may also be referred to as having a normal or low PR expression level. The lower the percentile, the lower the relative expression level.

Methods may involve the use of a normalized sample or control that is based on one or more cancer samples that are not from the patient being tested. This may be referred to as a reference sample in some embodiments. It is contemplated that a reference sample or a reference level of expression may be used in embodiments described herein. In some cases the reference level is an expression level or range of expression levels that qualifies a receptor as positive (+) or negative (−).

The methods involve treating a patient for breast or prostate cancer, which may include directly administering or providing a cancer therapy. In some embodiments, a practitioner or doctor may prescribe a cancer therapy that the patient administers to herself.

To achieve these methods, a doctor, medical practitioner, or their staff may retrieve a biological sample from a patient for evaluation. The sample may be a biopsy, such as a breast or prostate tissue or tumor biopsy. The sample may be analyzed by the practitioner or their staff, or it may be sent to an outside or independent laboratory. The medical practitioner may be cognizant of whether the test is providing information regarding the patient's level of HER-2, GR, ER, and/or PR expression or activity, or the medical practitioner may be aware only that the test indicates directly or indirectly that the test reflects that the patient has a particular phenotype or genotype or can be given a particular treatment regimen. Furthermore, the practitioner may know the patient's HER-2, ER, GR, and/or PR status, such as HER-2+ or HER-2−, ER+ or ER−, or GR+ or GR−, PR+ or PR−.

Other embodiments include a computer readable medium having software modules for performing a method comprising the acts of: (a) comparing glucocorticoid receptor data obtained from a patient's breast or prostate cancer sample with a reference; and (b) providing an assessment of estrogen receptor, glucocorticoid receptor, and/or progesterone receptor status to a physician for use in determining an appropriate therapeutic regimen for a patient. In further embodiments, the computer readable medium further comprises a software module for assessing estrogen receptor status of the patient's cancer sample.

Computer systems are also included. In some embodiments, they have a processor, memory, external data storage, input/output mechanisms, a display, for assessing glucocorticoid receptor activity, comprising: (a) a database; (b) logic mechanisms in the computer generating for the database a gene expression reference; and (c) a comparing mechanism in the computer for comparing the gene expression reference to expression data from a patient sample using a comparison model to determine a gene expression profile of the sample.

Other embodiments include an internet accessible portal for providing biological information constructed and arranged to execute a computer-implemented method for providing: (a) a comparison of gene expression data of one or more genes in a patient sample with a calculated reporter index; and (b) providing an assessment of gene activity or expression to a physician for use in determining an appropriate therapeutic regime for a patient.

In addition to compiling, collecting and or processing data related to gene status, methods, media and systems may also include the same embodiments with respect to data related to receptor status. Such aspects may be instead of or in addition to the aspects related to GR status or data.

It is contemplated that in methods described herein, breast or prostate cancer cells may undergo apoptosis following treatment set forth herein. Moreover, in some embodiments, the combination therapy described herein induces more apoptosis or kills or inhibits more cancer cells than treatment with just the anticancer treatment alone.

Use of the one or more compositions may be employed based on methods described herein. Other embodiments are discussed throughout this application. Any embodiment discussed with respect to one aspect of the invention applies to other aspects of the invention as well and vice versa. The embodiments in the Example section are understood to be embodiments o that are applicable to all aspects of the technology described herein. It is also contemplated that any compound or active ingredient may be specifically excluded in the methods and compositions of the disclosure.

By “gene” is meant any polynucleotide sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

As used herein, “treatment” or “therapy” is an approach for obtaining beneficial or desired clinical results. This includes: reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the disorder, shrinking the size of the tumor, decreasing symptoms resulting from the disease, increasing the quality of life of those suffering from the disease, decreasing the dose of other medications required to treat the disease, delaying the progression of the disease, and/or prolonging survival of patients.

The term “therapeutically effective amount” refers to an amount of the drug that may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “overexpress”, “overexpression”, “overexpressed”, “up-regulate”, or “up-regulated” interchangeably refer to a biomarker that is transcribed or translated at a detectably greater level, usually in a cancer cell, in comparison to a non-cancer cell or cancer cell that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis. Overexpression can be detected using conventional techniques for detecting mRNA (i.e., RT-PCR, PCR, hybridization) or proteins (i.e., ELISA, immunohistochemical techniques, mass spectroscopy). Overexpression can be 10%, 20%, 30%, 40%, 50%, 60%, 70%, 80%, 90% or more in comparison to a normal cell or cancer cell that is not associated with the worst or poorest prognosis. In certain instances, overexpression is 1-fold, 2-fold, 3-fold, 4-fold 5, 6, 7, 8, 9, 10, or 15-fold or more higher levels of transcription or translation in comparison to a non-cancer cell or cancer cell that is not associated with the worst or poorest prognosis.

“Biological sample” includes sections of tissues such as biopsy and autopsy samples, and frozen sections taken for histologic purposes. Such samples include breast or prostate cancer tissues, cultured cells, e.g., primary cultures, explants, and transformed cells. A biological sample is typically obtained from a mammal, such as a primate, e.g., human.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., breast), the size and type of the tumor, among other factors. Representative biopsy techniques include, but are not limited to, excisional biopsy, incisional biopsy, needle biopsy, and surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. Biopsy techniques are discussed, for example, in Harrison's Principles of Internal Medicine, 2005. Obtaining a biopsy includes both direct and indirect methods, including obtaining the biopsy from the patient or obtaining the biopsy sample after it is removed from the patient.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term consisting essentially of, as used herein with respect to compositions, is intended to mean that the active ingredients in the composition consist of only the active ingredients listed in the claims. Therefore, a composition consisting essentially of a GR modulator and a BET inhibitor, for example, would exclude any other active ingredients, but may include any other pharmaceutical excipients or carriers.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Endogenous BRD expression. Both parental and Enza-R LNCaP CWR-R1 cells express BRD4, BRD3 under normal growth conditions.

FIG. 2. Dex-mediated GR (NR3C1) target gene expression decreased with BETinhibition. LNCaP-EnzR cells were treated for three days of R1881 and enzalutamide (RE), after which cells stimulated with Dex (D) or Dex+JQ1. All conditions relative to RE. Changes all significant p<0.05 with no change in GR expression across conditions. Each series of bars from left to right corresponds to data from RE, RED, and RED-JQl.

FIG. 3. Dex-mediated cell viability significantly decreased with BETinhibition. LNCaP-EnzR cells were treated for three days of R1881 and enzalutamide (RE), concurrently with vehicle, Dex (D) or D+JQ1. Error bars=standard error of mean. The bars, from left to right, correspond to RE, RED, and RED-JQ 1.

FIG. 4. Effect of ABT-075 on AR/GR, BRD3/4 expression in Enza-R CRPC cell lines.

FIG. 5A-B. ABT-075 reduces GR target gene expression levels in CRPC. Cells treated with listed conditions for six hours. Top: CWR-R1 Enza-R. Bottom: LNCaP Enza-R. Left: ZBTB16. Right: FKBPS.

FIG. 6. BET Inhibition Delays CRPC Proliferation.

FIG. 7. Diagram illustrating metastatic tumor study.

### DETAILED DESCRIPTION OF THE INVENTION

Few targetable molecular drivers have been identified for Triple-negative breast cancer (TNBC) and thus standard treatment is limited to non-selective chemotherapy. Previous studies by the inventor had revealed that glucocorticoid receptor (GR) overexpression and accompanying increased GR transcriptional activity in TNBC is a potential target for improving TNBC therapeutic responses. GR overexpression occurs in about 30% of early-stage TNBC and contributes to chemotherapy-resistance through the ability of GR to activate the expression several potent EMT and anti-apoptotic genes, both directly and indirectly through GR-mediated chromatin remodeling. Bromodomian protein inhibitors (also referred to as BET inhibitors) have been shown to be cytotoxic in TNBC cell lines and in vivo models, although the exact mechanisms and biomarkers likely to predict tumor responsiveness are not clear. Bromodomains (BRDs) recognize acetylated lysine residues, such as those on the N-terminal tails of histones, and BRD-containing proteins are critical components for GR transcription. A well-known example of a BRD family protein required for glucocorticoid receptor (GR) transcriptional function is the BET (BRD and extra-terminal domain family) or BRD4 protein (3,4). BRD-containing proteins are expected to be disrupted by BET inhibitors (BETi) and thereby disrupt GR-mediated transcription and chromatin remodeling.

BETis may disrupt GR transcriptional activity in chemotherapy-resistant GR+ TNBC via inhibition of BRD proteins including BRD4, thereby abrogating the induction of GR-mediated anti-apoptotic gene expression and increasing sensitivity to chemotherapy. It is contemplated that BETi treatment of TNBC cell lines will alter the transcriptional activity of GR, and BETi treatment as well as GR modulation (such as inactivation or alteration of transcriptional activity) will make TNBC cells more susceptible to chemotherapy-induced cytotoxicity. It is also contemplated that inhibiting BET activity will lead to disruption of known GR-associated oncogenic and chemotherapy resistance pathways in TNBC and prostate cancer, thereby decreasing in vivo tumor growth compared to chemotherapy alone by increasing chemotherapy effectiveness.

### I. Hormone Receptor Status of Cancer

Intracellular receptors (IRs) form a class of structurally-related genetic regulators scientists have named “ligand dependent transcription factors” (R. M. Evans, Science, 240:889, 1988). Steroid receptors are a recognized subset of the IRs, including androgen receptor (AR), progesterone receptor (PR), estrogen receptor (ER), glucocorticoid receptor (GR), and mineralocorticoid receptor (MR). Regulation of a gene by such factors requires both the IR itself and a corresponding ligand, which has the ability to selectively bind to the IR in a way that affects gene transcription.

Naturally occurring as well as synthetic steroidal glucocorticoids (e.g., cortisol, cortisone, prednisolone, dexamethasone) have been widely used for over fifty years for the treatment of acute and chronic inflammatory and immune disorders. In particular, glucocorticoids have been prescribed for the treatment of rheumatoid arthritis, osteoarthritis, rheumatic fever, asthma, allergic rhinitis, systemic lupus erythematosus, chronic obstructive pulmonary disease, Crohn's disease, inflammatory bowel disease, and ulcerative colitis. However, the use of glucocorticoids is often associated with severe and sometimes irreversible side effects such as bone loss/osteoporosis, hyperglycemia, diabetes mellitus, hypertension, glaucoma, muscle atrophy, Cushing's syndrome, and psychosis.

Glucocorticoids exert their pharmacological effects by regulating gene transcription after the formation of a complex with the glucocorticoid receptor (GR). GR-glucocorticoid complex affects gene transcription by translocating to the nucleus after binding of the glucocorticoid where it acts as a dimer in binding to DNA glucocorticoid hormone response elements (GREs) in the promoter regions of particular genes. The GR-glucocorticoid/GRE complex then, in turn, activates (transactivation) or inhibits transcription of proximally located genes. Conversely, the GR-glucocorticoid complex may negatively regulate gene transcription by a process that does not involve binding to DNA. In this process, termed transrepression, following binding of the glucocorticoid, the complexed GR enters the nucleus where it acts as a monomer to directly interact (via protein-protein interaction) with other transcription factors, repressing their ability to induce gene transcription and thus protein expression.

Estrogen, mediated through the estrogen receptor (ER), plays a major role in regulating the growth and differentiation of normal breast epithelium (Pike et al. Epidemiologic Reviews (1993) 15(1):17-35; Henderson et al. Cancer Res. (1988) 48:246-253). It stimulates cell proliferation and regulates the expression of other genes, including the progesterone receptor (PR). PR then mediates the mitogenic effect of progesterone, further stimulating proliferation (Pike et al., 1993; Henderson et al., 1988). The molecular differences between estrogen receptor (“ER”) negative and ER positive tumors are significant in light of clinical observations which indicate that the nature and biological behavior of ER positive and ER negative tumors are distinct even in the absence of hormonal therapy. For example, ER negative cancers tend to recur sooner and show a different rate of recurrence in distant organ sites compared to ER positive tumors. Clinical observations and molecular profiling data suggest that tumors not expressing both ER and PR represent a different clinical entity in terms of chemotherapy responsiveness. (Colleoni et al., Annals of Oncology 11(8):1057 (2000)). Thus, ER negative and ER positive breast cancers are two distinct disease entities rather than phenotypic variations of the same disease.

### II. Biomarkers and Evaluating Levels of Biomarkers

Biomarkers for prognosing human breast or prostate cancer patients have been identified. They include estrogen receptor (ER) in combination with the activity of the glucocorticoid receptor (GR) activity. Androgen receptor (AR) can also be used as a cancer biomarker. It is contemplated that these biomarkers may be evaluated based on their gene products. In some embodiments, the gene product is the RNA transcript. In other embodiments, the gene product is the protein expressed by the RNA transcript. In still another embodiment is the evaluation of surrogate genes or gene targets of AR, ER, GR, or ER and GR.

In certain aspects a meta-analysis of expression or activity can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a marker gene or activity. Normalization of the expression of a marker gene is preferably achieved by dividing the expression level of the individual marker gene to be normalized by the respective individual median expression of this marker genes, wherein said median expression is preferably calculated from multiple measurements of the respective gene in a sufficiently large cohort of test individuals. The test cohort preferably comprises at least 3, 10, 100, 200, 1000 individuals or more including all values and ranges thereof. Dataset-specific bias can be removed or minimized allowing multiple datasets to be combined for meta-analyses (See Sims et al. BMC Medical Genomics (1:42), 1-14, 2008, which is incorporated herein by reference in its entirety).

The calculation of a meta-gene expression value is performed by: (i) determining the gene expression value of at least two, preferably more genes (ii) “normalizing” the gene expression value of each individual gene by dividing the expression value with a coefficient which is approximately the median expression value of the respective gene in a representative breast cancer cohort (iii) calculating the median of the group of normalized gene expression values.

A gene shall be understood to be specifically expressed in a certain cell type if the expression level of said gene in said cell type is at least 2-fold, 5-fold, 10-fold, 100-fold, 1000-fold, or 10000-fold higher than in a reference cell type, or in a mixture of reference cell types. Reference cell types include non-cancerous breast or prostate tissue cells or a heterogenous population of breast or prostate cancers.

In certain algorithms a suitable threshold level is first determined for a marker gene. The suitable threshold level can be determined from measurements of the marker gene expression in multiple individuals from a test cohort. The median expression of the marker gene in said multiple expression measurements is taken as the suitable threshold value.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

“A sufficiently large number”, in this context, means preferably 30%, 50%, 80%, 90%, or 95% of the marker genes used.

In certain aspects, the determination of expression levels is on a gene chip, such as an Affymetrix™ gene chip.

In another aspect, the determination of expression levels is done by kinetic real time PCR.

In certain aspects, the methods can relate to a system for performing such methods, the system comprising (a) apparatus or device for storing data on the ER or nodal status of the patient; (b) apparatus or device for determining the expression level of at least one marker gene or activity; (c) apparatus or device for comparing the expression level of the first marker gene or activity with a predetermined first threshold value; (d) apparatus or device for determining the expression level of at least one second marker gene or activity; and (e) computing apparatus or device programmed to provide a unfavorable or poor prognosis if the data indicates a negative ER status and an increased or decreased expression level of said first marker gene or activity (e.g., GR expression or activity) with the predetermined first threshold value and, alternatively, the expression level of said second marker gene is above or below a predetermined second threshold level.

The person skilled in the art readily appreciates that an unfavorable or poor prognosis can be given if the expression level of the first marker gene with the predetermined first threshold value indicates a tumor that is likely to recur or not respond well to standard therapies.

The expression patterns can also be compared by using one or more ratios between the expression levels of different cancer biomarkers. Other suitable measures or indicators can also be employed for assessing the relationship or difference between different expression patterns.

The GR nucleic acid and protein sequences are provided in GenBank accession number AY436590. The ER nucleic acid and protein sequences are provided in GenBank accession number NG_008493. The content of all of these GenBank Accession numbers is specifically incorporated herein by reference as of the filing date of this application.

The following biomarkers are provided for implementation with embodiments discussed herein. All of them designate nucleic acid sequences for the particular gene identifier. Nucleic acid sequences related to these gene designation can be found in the Genbank sequence databases. Additional biomarkers include the MCL1, SAP30, DUSP1, SGK1, SMARCA2, PTGDS, TNFRSF9, SFN, LAPTMS, GPSM2, SORT1, DPT, NRP1, ACSLS, BIRC3, NNMT, IGFBP6, PLXNC1, SLC46A3, C14orf139, PIAS1, IDH2, SERPINF1, ERBB2, PECAM1, LBH, ST3GAL5, IL1R1, BIN1, WIPF1, TFPI, FN1, FAM134A, NRIP1, RAC2, SPP1, PHF15, BTN3A2, SESN1, MAP3K5, DPYSL2, SEMA4D, STOM, MAOA, AKAP1, AREG, ARHGEF26, BIRC3, CA12, CALCR, CDC42EP3, CYP24A1, DEPTOR, DOCK4, DUSP6, FGF18, FOS, GAD1, GREB1, IL6R, IL6ST, KAZN, KCNJ8, KDM4B, KIAA0226L, KLF9, LAMA3, MAFB, MYC, NR5A2, PER1, PHLDAL PSCA, RGS2, RHOBTB1, SGK1, SNAI2, SOCS2, SYBU, TBC1D8, TGFA, WIPF1, WWC1, ALDH1A3, CXCL12, LRRC15, LY6H, NR4A2, PDZK1, PPIF. SLC22A4, RNF43, ARL14, CD44, CYP1A1, DDX10, EGR3, EMP1, FJX1, HCK, HEG1, HEY2, PTGES, RAB31, RARA, SIM1, SLC26A2, TMEM120B, TNFRSF11B, TRPC6, DIRAS2, KRT13, LRP4, PTGER4, RET, RGCC, SEMA3B, SERPINB9, SLC47A1, SUV39H2, RAPGEFL1, MICB, HS3ST3A1, HSPB8, IGFBP4, JAK2, KIT, LEF1, LINC00341, MAFF MYBL1, NPY1R, NPY5R, PGR, PLAC1, and PMAIP1 genes.

The expression levels of cancer biomarkers can be compared to reference expression levels using various methods. These reference levels can be determined using expression levels of a reference based on all cancer patients or all breast or prostate cancer patients determined to be ER+ and/or ER−. Alternatively, it can be based on an internal reference such as a gene that is expressed in all cells. In some embodiments, the reference is a gene expressed in breast or prostate cancer cells at a higher level than any biomarker. Any comparison can be performed using the fold change or the absolute difference between the expression levels to be compared. One or more breast or prostate cancer biomarkers can be used in the comparison. It is contemplated that 1, 2, 3, 4, 5, 6, 7, 8, and/or 9 biomarkers may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are by normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments of the invention.

Any biological sample from the patient that contains breast or prostate cancer cells may be used to evaluate the expression pattern of any biomarker discussed herein. In some embodiments, a biological sample from a breast or prostate tumor is used. Evaluation of the sample may involve, though it need not involve, panning (enriching) for cancer cells or isolating the cancer cells.

### A. Nucleic Acids

Screening methods based on differentially expressed gene products are well known in the art. In accordance with one aspect of the present invention, the differential expression patterns of breast or prostate cancer biomarkers can be determined by measuring the levels of RNA transcripts of these genes, or genes whose expression is modulated by the these genes, in the patient's breast or prostate cancer cells. Suitable methods for this purpose include, but are not limited to, RT-PCR, Northern Blot, in situ hybridization, Southern Blot, slot-blotting, nuclease protection assay and oligonucleotide arrays.

In certain aspects, RNA isolated from breast or prostate cancer cells can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes for a breast or prostate cancer biomarker can be prepared from the gene sequence or obtained through commercial sources, such as Affymatrix. In certain embodiments the gene sequence is identical or complementary to at least 8 contiguous nucleotides of the coding sequence.

Sequences suitable for making probes/primers for the detection of their corresponding breast or prostate cancer biomarkers include those that are identical or complementary to all or part of genes described herein. These sequences are all nucleic acid sequences of breast or prostate cancer biomarkers.

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). Preferably, each probe/primer has relatively high sequence complexity and does not have any ambiguous residue (undetermined “n” residues). The probes/primers preferably can hybridize to the target gene, including its RNA transcripts, under stringent or highly stringent conditions. In some embodiments, because each of the biomarkers has more than one human sequence, it is contemplated that probes and primers may be designed for use with each on of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In another embodiment, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one embodiment, quantitative RT-PCR (such as TaqMan, ABI) is used for detecting and comparing the levels of RNA transcripts in cancer samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products preferably are carried out when the PCR reactions are in the linear portion of their curves. In addition, relative concentrations of the amplifiable cDNAs preferably are normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes one or more internal PCR standards. The internal standard may be an abundant housekeeping gene in the cell or it can specifically be GAPDH, GUSB and (3-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of cancer biomarkers in cancer cells. The probes suitable for detecting the corresponding cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array of the present invention can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm².

Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods of the invention.

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; WO 03020898; WO 03040410; WO 03053586; WO 03087297; WO 03091426; W003100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In one embodiment, nuclease protection assays are used to quantify RNAs derived from the cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

### B. Proteins and Polypeptides

In other embodiments, the differential expression patterns of cancer biomarkers can be determined by measuring the levels of polypeptides encoded by these genes in cancer cells. Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the cancer biomarker genes described herein.

One example of a method suitable for detecting the levels of target proteins in peripheral blood samples is ELISA. In an exemplifying ELISA, antibodies capable of binding to the target proteins encoded by one or more cancer biomarker genes are immobilized onto a selected surface exhibiting protein affinity, such as wells in a polystyrene or polyvinylchloride microtiter plate. Then, cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another ELISA embodiment, the cancer cell samples containing the target proteins are immobilized onto the well surface and then contacted with the antibodies of the invention. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 49° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Various protocols for conducting RIA to measure the levels of polypeptides in cancer cell samples are well known in the art.

Suitable antibodies for this invention include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In other aspects, the levels of polypeptides in samples can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

### III. Cancer Therapy

Certain embodiments are directed to methods of treating breast or prostate cancer based on the AR, ER, GR, HER-2 and/or GR status of the cancer tissue. Further embodiments relate to treating prostate cancer such as GR+ prostate cancers. In some embodiments, the hormone receptor status is determined based on the expression of a hormone receptor such as the estrogen receptor (ER) in combination with the glucocorticoid receptor (GR).

Embodiments concern glucocorticoid receptor mixed agonists and modulators. In some embodiments, the glucocorticoid receptor mixed agonist/antagonist or modulator is RU-486, RU-43044, RU-38486, CP-409069 ORG 214007, ORD ZK-216348, CORT 125134, GSK 9027, AL-438, ZK 245186, CmdA, BI115, Quinol-4-ones, LGD5552, ZK 216348, GS 650394, CORT 01 13083, CORT 001 12716 or analogs or metabolites thereof Also included are steroidal GR modulators which include 11-Monoaryl and 11,21 Bisaryl steroids, 11Beta-Aryl conjugates of mifepristone, and non-steroidal modulators, including octahydrophenanthrenes, spirocyclic dihydropyridines, triphenyl methanes (e.g. AL082D06), chromens, dibenzyl analines, dihydroqinolones, pyrimidine diones, fused azedecalins (e.g. 113176 and CORT 108297), and indole sulfonamides. Structurally-related compounds that also are GR antagonists or modulators include diaryl ethers, aryl pyrazolo azadecalins, phenanthrenes, dibenzol [2.2.2]cycloctaines and derivatives, dibenzoclyclohepatnes and their derivatives, dibenzyl anilinesulfonamides and their derivatives, dihetero(aryl) pentanol, chromene derivatives, Azadecalins, aryl quinolones, and 11-aryl, and 16-hydroxy steroids.

In some embodiments, the GR modulator is one that alters the transcriptional acitivy of the glucocorticoid receptor. Altering the transcriptional activity may be reducing or abolishing the expression of GR-target genes in the cancer cells. For example and in some embodiments, the GR modulator abrogates the induction of GR-target genes in cancer cells. In some embodiments, the GR modulator reduces or eliminates the expression of anti-apoptotic genes in the cancer cells.

In some embodiments, a chemotherapeutic agent is administered to the cells or patient. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), raloxifene, taxol, gemcitabine, navelbine, farnesyl-protein transferase inhibitors, transplatinum, 5-fluorouracil, vincristin, vinblastin and methotrexate, or any analog or derivative variant of the foregoing.

Suitable therapeutic agents include, for example, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

Certain chemotherapeutics are well known for use against breast cancer. These breast cancer chemotherapeutics are capecitabine, carboplatin, cyclophosphamide (Cytoxan), daunorubicin, docetaxel (Taxotere), doxorubicin (Adriamycin), epirubicin (Ellence), fluorouracil (also called 5-fluorouracil or 5-FU), gemcitabine, eribulin, ixabepilone, methotrexate, mitomycin C, mitoxantrone, paclitaxel (Taxol), thiotepa, vincristine, vinorelbine.

Other chemotherapeutics such as docetaxel, cabazitaxel, mitoxantrone, abiraterone, prednisone, radium-223, sipuleucel-T, mitoxantrone, bicalutamide, flutamide, nilutamide, ketoconazole, and low-dose corticosteroids can be used to treat the prostate cancer.

The methods may further comprise treatment with a chemotherapeutic as described herein or with other conventional cancer therapies. Conventional cancer therapies include one or more selected from the group of chemical or radiation based treatments and surgery.

With respect to the combination therapy described herein, various combinations may be employed, for example BET inhibitor is “A” and the GR modulator and/or chemotherapeutic agent is “B”:
A/B/A B/A/B B/B/A A/A/B A/B/B B/A/A A/BBB B/A/B/B
BBB/A B/B/A/B A/A/B/B AB/AB A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

Radiation therapy that causes DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted,” “administered,” and “exposed,” when applied to a cell, are used herein to describe the process by which a therapeutic construct and a chemotherapeutic, GR modulator, or BET inhibitor are delivered to a target cell or are placed in direct juxtaposition with the target cell. To achieve cell killing or stasis, both or all agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg (or any range derivable therein). A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500 mg/kg/day (or any range derivable therein).

Alternative cancer therapy include any cancer therapy other than surgery, chemotherapy and radiation therapy in the present invention, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into an individual's cells and tissues to treat a disease. Antisense therapy is also a form of gene therapy in the present invention. A therapeutic polynucleotide may be administered before, after, or at the same time of a first cancer therapy. Delivery of a vector encoding a variety of proteins is encompassed within the invention. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DRS/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### IV. Pharmaceutical Compositions

Embodiments include methods for treating cancer. Administration of the compositions will typically be via any common route. This includes, but is not limited to oral, parenteral, orthotopic, intradermal, subcutaneous, intramuscular, intraperitoneal, intranasal, or intravenous injection. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%. In some embodiments, the compositions are administered orally.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of a pharmaceutical composition are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2 day to twelve week intervals, more usually from one to two week intervals. The course of the administrations may be followed by assays for GR activity, cell survival, or BET activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The compositions of the disclosure can be formulated for parenteral administration, e.g., formulated for injection via the intravenous, intradermal, intramuscular, sub-cutaneous, or even intraperitoneal routes. In some embodiments, the composition is administered by intravenous injection. The preparation of an aqueous composition that contains an active ingredient will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### V. Kits

Certain aspects of the present invention also encompass kits for performing the methods of the disclosure, such as detection of or diagnosis of TNBC and/or GR status of the patient. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

In a particular aspect, these kits may comprise a plurality of agents for assessing the differential expression of a plurality of biomarkers, for example, GR, ER, HER-2, and/or PR, wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Combination Therapy

TNBC, GR+ cells can be treated with combination therapies comprising a GR modulator and a BET inhibitor; a BET inhibitor and a chemotherapeutic; a BET inhibitor a GR modulator, and a chemotherapeutic, or they are treated with each agent alone. It is contemplated that the combination of the BET inhibitor and the GR modulator or chemotherapeutic or the combination of all three agents with reduce growth, increase apotosis, or increase necrosis more than either agent alone. It is contemplated that the combination therapy will show a synergistic effect.

### Example 2

### Preclinical Investigation of a BET Inhibitor in the Treatment of High Glucocorticoidreceptor (GR) Expressing Enzalutamide-Resistant (Enza-R) Castration-Resistant Prostate Cancer (CRPC)

Castration-resistant prostate cancer (CRPC) is a lethal malignancy affecting thousands of men yearly, in the United States alone. Although potent hormonal therapies, including the androgen receptor (AR) antagonist enzalutamide are clinically effective, their benefit is typically temporary, with mortality subsequent to progression on these medications 2-3 years at most. The inventors have discovered that in addition to sustained AR signaling, a similar hormone receptor, the glucocorticoid receptor (GR) may play a role CRPC progression. Others have shown that in other cell times, BET bromodomain proteins interact with GR to fascilitate transcription. It is contemplated that BET bromodomain inhibitors such as ABT-075, for example, can act as a novel method of dual AR and GR concurrent inhibition in clinically relevant GR-expressing preclinical CRPC models and thus to provide the initial evidence that BET bromodomain inhibitors should be developed in GR− overexpressing (GR+) enzalutamide resistant (Enza-R) CRPC. This example describes methods to 1) identify the GR/AR and BRD4-associated superenhancers involved in Enza-R cell survival, 2) determine if BET bromodomain inhibitors diminish GR-mediated gene regulation in Enza-R CRPC, and 3) demonstrate that BET bromodomain inhibitors prolong GR+ Enza-R CRPC survival in vivo.

Therapies targeting clinical resistance to enzalutamide (Enza-R) is a colossal unmet medical need. CRPC is a lethal disease that causes incalculable morbidity and leads to mortality in approximately 25,000 men in the United States alone each year. Enzalutamide, a highly effective androgen receptor (AR) antagonist is approved by the FDA for the treatment of castration-resistant prostate cancer (CRPC); however, the survival improvement with the drug is limited, with patients dying from Enza-R CRPC within 2-3 years. The current management strategies for this lethal clinical entity are limited to chemotherapy and in select cases, the radionuclide Radium-223. In both cases, the magnitude of benefit is minimal. There are no currently FDA-approved molecularly targeted therapies focused on de novo or acquired Enza-R biology in CRPC.

Enza-R CRPC can be driven through sustained nuclear hormone signaling. There are multiple mechanisms that can lead to Enza-R. The inventors have developed a series of Enza-R CRPC cell lines and have found that sustained nuclear hormone signaling is a common feature of Enza-R CRPC1. The majority of nuclear hormone research in prostate cancer, including in Enza-R has focused on the AR. The development Enza-R tumors expressing AR splice variants that do not require AR ligand binding or are active despite enzalutamide due to mutations in the ligand biding domain is established in both the preclinical and clinical settings; however, the inventors and others have recently shown that there may be another nuclear hormone signaling pathway implicated in Enza-R. Overexpression and/or activation of the glucocorticoid receptor (GR), a distinct nuclear hormone receptor structurally similar to AR, can compensate for AR blockade and lead to Enza-R cancer cell survival. Developing therapies that specifically target these two nuclear hormone receptors, AR and GR, in tandem could have significant clinical impact through suppression of two key mediators of CRPC progression.

Bromodomain inhibition can perturb AR and GR signaling. As sustained nuclear hormone signaling can be a powerful mechanism of enabling Enza-R, it is critical that one identify and validate therapeutic targets that can mitigate this tumor cell survival strategy. BET-family proteins, such as BRD4 have a known role in facilitating nuclear receptor-mediated transcription. It has recently been shown that BRD4 can associate with both the AR and GR transcription factors to enable their function at DNA super-enhancer regions. The affect of bromodomain inhibition on GR function in CRPC is not known. It is contemplated that the BET bromodomain inhibitors will diminish Enza-R CRPC progression in relevant GR/AR-expressing preclinical CRPC models through dual disruption of both GR and AR transcriptional activity. Should this novel hypothesis be supported, this work will provide critical initial evidence that BET inhibitors should be developed clinically in GR− overexpressing Enza-R CRPC. Given the exceptionally poor prognosis for Enza-R CRPC and the lack of molecularly targeted therapies for this clinical state, BET inhibitors have real potential to have a major impact against a terrible disease. In addition, this work may potentially identify new BET-inhibited therapeutic targets, driven by nuclear hormone signaling for future development.

The central hypothesis is that GR-expressing Enza-R CRPC is driven by sustained nuclear hormone signaling, and that BET inhibitors will disrupt GR-mediated prosurvival gene regulation and delay metastatic CRPC Enza-R progression through BET-inhibition. To investigate the role of BET inhibitors on CRPC, cell lines such as the Enza-R CRPC cell line and the LNCaP-EnzaR cell line can be used. Although the parental cell line does not express appreciable levels of GR, the LNCaP-EnzaR line induces high levels of GR expression during the development of Enza-R and displays a metastatic phenotype in vivo. Of note, its AR contains a point mutation in its ligand binding domain allowing it to bind non-androgen nuclear hormones, but it has no detectable AR ligand binding domain lacking splice variants (e.g. AR-V7). Its high GR expression and lack of AR splice variants is ideal as it allows for the specific testing of BET inhibitors on GR affects without any confounding affect on the splice variants not antagonized by enzalutamide. The CWR-R1-EnzaR line is a biologically distinct, de novo castration-resistant cell line, which expresses high levels of GR and also expresses AR splice variants. Thus, these two cell lines represent varied AR biology, are both Enza-R and both express high levels of GR.

The experiments described herein can be used to identify DNA enhancer elements through which GR and BRD4 cooperate to enable sustained cell survival signaling subsequent to AR blockade with enzalutamide. Activation of GR can compensate for AR transcriptionally and facilitate Enza-R. Using several different human prostate cancer cell lines that endogenously express both AR and GR, the inventors have demonstrated that the activity of GR is AR-context dependent; when AR is active GR activation can be growth inhibitory, whereas when AR is sufficiently blocked, GR activation enhances prostate cancer cell proliferation. Furthermore, subsequent to AR inhibition with enzalutamide, GR can activate transcription of selected pro-survival genes previously driven by AR and directly bind DNA elements previously occupied by the AR.

The inventors recently discovered that within PC cells, activated GR can compensate transcriptionally for a large set of genes. Using next-generationdeep sequencing of RNA (RNA-seq), the inventors showed show that in the LAPC4 cell line, 442 genes, and in the CWR-22Rv1 cell line 272 genes are induced by AR activation, reversed with AR antagonism and reinduced by GR activation. In a companion experiment using chromatin immunoprecipitation followed by deep sequeicing (ChIP-seq) in LAPC4 cells, the addition of dexamethasone to activate the GR leads to ˜700 new unique GR binding sites, approximately 70 of these new sites are within 5 kilobase (kb) of the transctiption start site of known genes. This argues that the majority of GR chromatin binding and subsequent gene regulation is through regulation at more distant enhancer sites rather then through direct promoter site binding. As bromodomain proteins such as BRD4 cooperate with known transcription factors, including the GR, at such enhancers to enable gene regulation, the inventors sought to establish whether BRD4 and GR cooperate to regulate pro-survival genes in Enza-R CRPC.

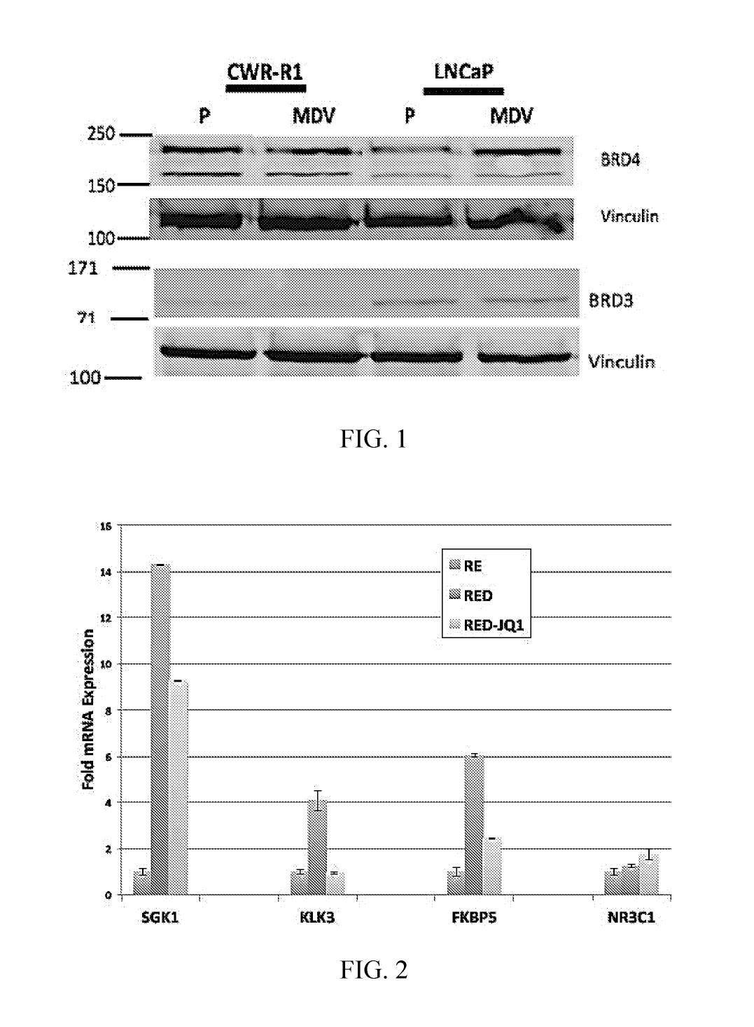

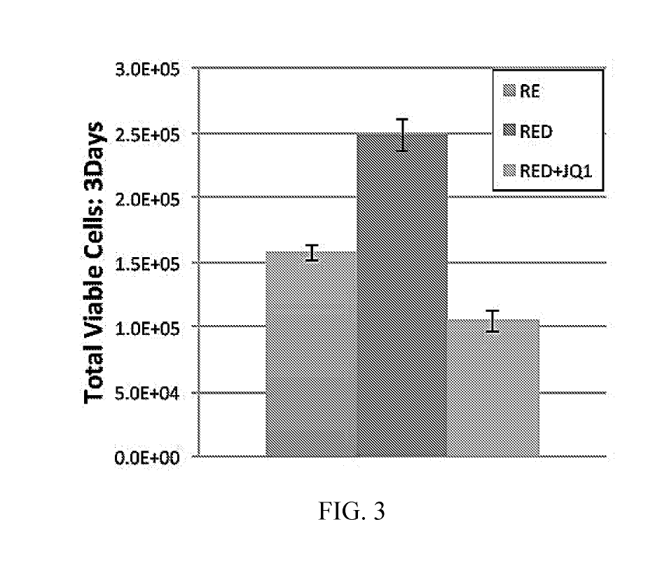

The inventors have also verified that the principal BET-BRD proteins inhibited by a BET inhibitor (ABT-075), BRD4 and BRD3 are expressed within the Enza-R CRPC cell lines. As shown in FIG. 1, both BRD proteins are highly expressed within the cell lines. It was next sought to explore whether for specific, known GR-regulated in PC cells genes (KLK3, SGK1, FKBPS) the BET inhibitor tool compound JQ1 was able to decrease GR mediated gene expression. For all three genes tested, BET-inhibition significantly countered, to different extents, the gene expression increases mediated by GR (FIG. 2).

The extent to which GR and the BET BRD proteins, such as BRD4 interact and cooperate to regulate genes in Enza-R CRPC is unknown. One goal of the experiments described herein is to determine what genes that are regulated by GR subsequent to AR blockade are facilitated by BRD's. The Enza-R LNCaP line can be utilized for these experiments as it is highly metastatic in vivo, expresses GR, yet does not express ligand independent AR splice variants that may not be inhibited by enzalutamide but could be inhibited by BET inhibitors. The second cell line can be used to validate the findings. The cells can be cultured in vitro in phenol red free, nuclear hormone deprived media supplemented with enzalutamide (10 μM) along with AR agonist R1881 (1 nM) to mimic the condition found in Enza-R CRPC patients where there is small amount of androgen and an abundance of enzalutamide in the serum. The GR agonist dexamethasone (100 nM) can then be added with and without concurrent BET inhibitors such as ABT-075 (100 nM) to the cells for 6 hours. The six hour time point was chosen as a large number of genes are GR regulated at this time point; significantly more genes at this time point then at 2 hours (data not shown). RNA can be collected in biological triplicates. Following library preparation techniques known in the art, RNA expression analysis in these conditions can be performed utilizing Illumina Solexa Next-Gen sequencing technology (RNA-seq). A cohort of the differentially regulated genes can be validated by qRT-PCR in both the LNCaP-EnzaR and in the second CWR-R1-EnzaR cell line.

Differences in gene expression between conditions will be assessed using three distinct algorithms. Genes with average fold changes >1.5, common to all algorithms, will be required for a gene to be considered significantly up- or down-regulated. The ratios of gene expression change (fold change ratios) between the Dex and Dex+BETi conditions will be calculated to determine antagonism of GR-mediated transcription with the BETi. Assuming 5% of the genes are truly differentially expressed, for 90% power at an false discovery rate (FDR) of 0.05, a significance level of α=0.0025 will be required.

Further experiments described below can be done to show direct and cooperative binding of GR and BRD4 at enhancers of GR regulated, BET inhibitor-inhibited genes in Enza-R CRPC. The LNCaP-EnzaR cell lines can be cultured in vitro as described previously with and without BET inhibitor treatment. DNA can be collected and cross-linked, sonicated and BRD4 and GR-chromatin immunoprecipitation (ChIP), in two separate IP's, can be conducted. Deep sequencing of reverse cross-linked DNA can them be performed using Illumina Solexa Next-Gen sequencing technology, for example. Reads from deep sequencing will be aligned with the human genome and the corresponding input DNA reads can then be read into Model-based analysis of ChIP-Seq (MACS version 2.0) program for “peak” calling of significantly bound chromatin regions. A false-discovery rate (FDR) <=0.05 will be used to determine the significant GR binding regions (GBRs) and BRD4 binding regions (BRD4-BRs). GBRs and BRD4-BRs can then be compared to determine to what extent the two binding regions overlap. The transcriptional start site (TSS) of genes within 200kB of these overlapping bound GBRs and BRD4-BRs can be identified, signifying “co-bound genes”. Binding regions between the BET-inhibited conditions can also be compared to determine if BET inhibition disrupts BRD4 and GR binding. As a subsequent experiment, ChIPre-ChIP (first ChIP for GR and then ChIP the same DNA for BRD4 before reversing the crosslinkage) followed by targeted quantitative PCR of a set of co-bound genes can be used to determine if the two proteins interact simultaneously at the same regions of DNA. Finally, these co-bound genes can be compared to the BET-inhibited, GR-regulated genes identified in previous experiments described herein. In this way, a robust list of Enza-R genes with direct, coordinated regulation by BRD4 and GR that can be inhibited with BET inhibitors can be obtained.

It is contemplated that the experiments described herein will demonstrate that BET inhibititors such as ABT-075 can globally diminish AR/GR proliferative or pro-survival gene expression. It is anticipated that the inventors will discover whether, and to which genes BRD4 and GR bind concurrently and co-regulate, and determine to what extent BET-inhibition interferes with these actions. Finally, it is expected to identify a novel cohort of AR/GR regulated genes inhibited by BET-inhibition for further prioritization as future therapeutic targets in GR+ Enza-R CRPC. It is possible that analysis will yield too few genes bound by both GR and BRD4. In this case, the distance from TSS to capture can be increased for more distal enhancers. If too many regulated genes are identified, the stringency may be increased to 1.75 fold.

The experiments described below can be used to determine whether BET inhibitors can prolong metastatic GR+ Enza-R CRPC survival in vivo. As discussed above, one potential mechanism for Enza-R CRPC progression is sustained proliferation/cell survival imparted through GR signaling. These experiments may provide evidence that BET inhibition can diminish Enza-R progression through blockade of both AR and GR signaling. The LNCaP-EnzaR and CWR-R1-EnzaR cell lines are highly metastatic to bone and other organs and are excellent in vivo therapeutic models. Subsequent to intracardiac injection of these luciferase expressing Enza-R cell lines into male, castrated immunocompromised mice, the inventors have demonstrated significant metastatic colonization that can be followed with luciferase live animal imaging and shortened animal survival in comparison to parental cell lines.

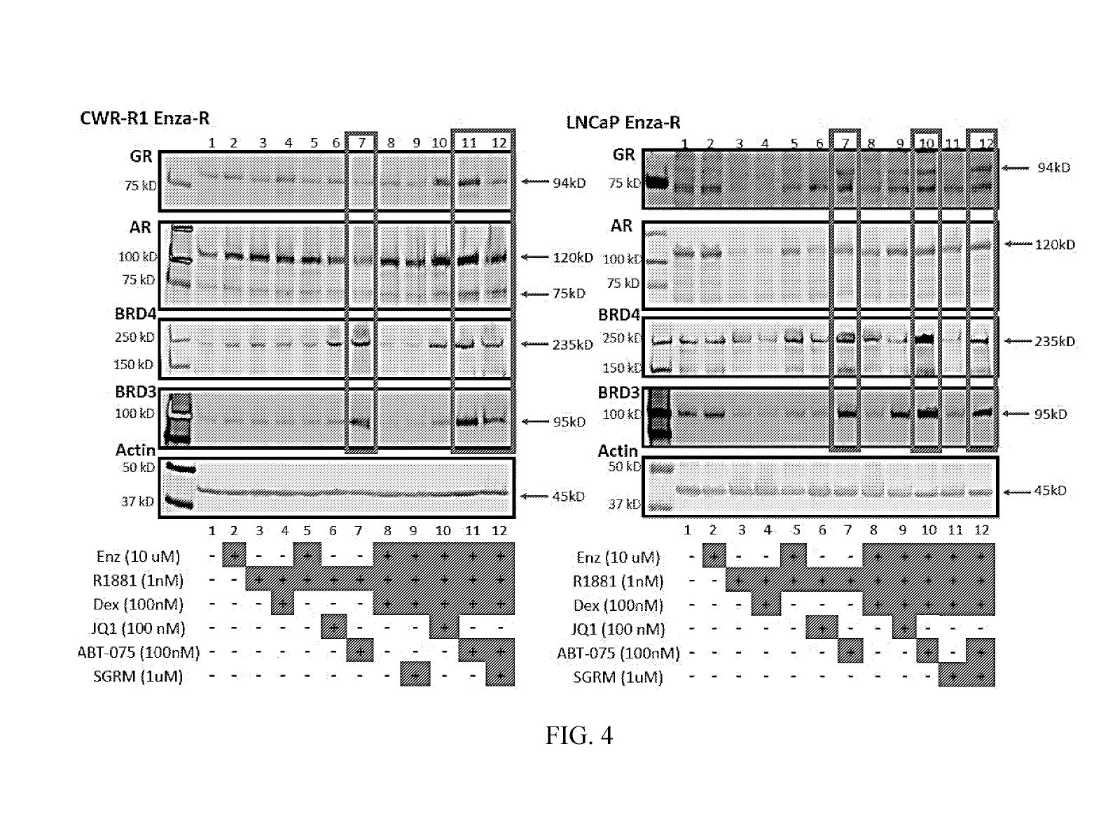

BET-inhibition decreases Enza-R survival in vitro despite dexamethasone. It has been reported that the BET inhibitor JQ1 (100-500 nM) can decrease Enza-R cell survival, potentially through inhibition of ligand independent AR splice variants. As a preliminary experiment to understand if BET inhibition can diminish GR-mediated Enza-R and to justify further in vivo interrogation with BET inhibitors, LNCaP-EnzaR cells, which do not contain AR splice variants, were treated with dexamethasone (100 nM) with and without JQ1 (500 nM) in the context of R1881 (1 nM) and enzalutamide (10 μM). As shown in FIG. 3, after 3 days of treatment, Dex GR activation increases viable cell numbers, which is decreased with JQ1.

Described herein are experiments to test the ability of BET inhibitors to decrease metastatic progression and prolong survival in our Enza-R models. Both Enza-R CRPC cell lines will be utilized to provide increased heterogeneity of Enza-R biology. In order to decipher to what extent BET inhibitor treatment effect is due to inhibition of GR activity and/or AR activity, different combinations of treatment will be necessary (Table 1). Enzalutamide treatment with infused diet (30 mg/kg13), will block AR activity and is safe and well tolerated in combination with a BET inhibitor such as ABT-075. ABT-075 can be dosed at 1 mg/kg oral gavage daily. Although there are GR antagonists available, the potential drug-drug interactions with ABT-075 are unknown. Thus, in order to specifically and selectively diminish GR activity, prior to inoculation, the cell lines will be engineered to express a doxycycline-inducible GR-targeted shRNA.

TABLE 1
In vivo treatment conditions with key outcome to test per condition

| Condition | Outcome/Question |
|---|---|
| 1 Control diet | CRPC progression baseline |
| 2 Enza | Enza-R progression baseline |
| 3 Enza + GR-KD | Dual AR/GR inhibition control |
| 4 Enza + ABT-075 | Does BET-inhibition prolong Enza-R |
| | progression? |
| 5 Enza + GR-KD + ABT-075 | Does BET-inhibition provide additive |
| | benefit beyond AR/GR |
| | blockade and vice versa? |
| 6 Control diet + ABT-075 | Is BET-inhibition sufficient to provide |
| | AR and GR inhibition? |

As described previously, metastatic CRPC tumors can be formed subsequent to intracardiac inoculation of SCID immunocompromised male castrated mice. Animals demonstrating metastatic colonization with weekly luciferase can be randomized into six different treatment arms as described in Table 1. Treatment will continue until endpoint, which is defined by intolerable tumor volume related symptoms such as weight loss, poor animal self-care, paralysis (from spine metastasis) or breathing difficulties, as outlined within the animal protocol. Metastatic burden can be calculated from bioluminescence imaging weekly and compared between conditions. Animal survival can be calculated using a Kaplan-Meier curve. The primary analysis will be to compare enzalutamide+AB-075 to enzalutamide treatment alone with respect to enzalutamide resistant CRPC survival. As described in Table 1, the other conditions will serve as controls.

Understanding that BET inhibitors may act through multiple mechanisms, including but not limited to GR signaling inhibition, the primary goal of this experiment will be to perform immunohistochemical (IHC) analysis for GR expression of metastatic tumors at various timepoints post metastatic initiation and treatment. It has previously shown that LNCaP CRPC tumors express high GR; however the GR expression is heterogeneous within xenograft tumors with very high expression in focal areas. Should the hypothesis that BET inhibitors will decrease viability through inhibition of GR, it is anticipated that over time, treatment with BET inhibitors will limit the outgrowth of GR expressing tumor cells; at endpoint, there should be less GR expressing tumor cells then at initiation of treatment. Metastatic LNCaP-EnzR tumors can be initiated as described previously. Mice can be euthanized and tumors extracted for downstream IHC analysis prior to treatment, after 7 days of treatment and at endpoint within each treatment cohort. Tumors can be fixed, decalcified if bone, paraffin embedded and sections stained for GR, AR, the proliferation marker Ki-67 and with hematoxylin and eosin. IHC expression can be assessed using a 0-3+ intensity multiplied by percentage positive stain scoring system algorithm. The mean scores for each marker can be calculated across 5 (or more if more then one metastases extracted) extracted tumors and compared between conditions.

The experiments described herein will test the hypothesis that BET inhibitors will be effective in vivo in inhibiting GR-mediated Enza-R. GR/AR expressing clinically relevant prostate cancer cell lines that develop lethal metastatic disease can be utilized. It is expected that BET inhibitors will delay Enza-R significantly in the cell lines. Notably, through combinatorial treatment cohorts, to what extent that effect is AR mediated or GR mediated and to what extent inhibition of these targets (AR, GR, BET BRDs) may offer additive benefit can be determined. By interrogating metastatic tumors ex vivo, it is anticipated that endpoint Enza-R metastases will have less GR expression in the BET inhibitor-treated cohorts implying selection for Enza-R driven by other mechanisms beyond GR signaling. It is possible that neither GR nor AR expression will change in tumors with BETi treatment. That would be an interesting finding and the tumors could be investigated for mRNA expression of target genes.

### Example 3

### Preclinical Investigation of ABT-075 in the Treatment of High Glucocorticoid Receptor Expressing Enzalutamide-Resistant Castration-Resistant Prostate Cancer

Prostate cancer is canonically driven by androgen receptor (AR) signaling. It is the third leading cause of cancer death among men in the United States. AR promotes differentiation of the prostate in normal development, but its transcription factor function is redirected to drive a malignant phenotype in prostate cancer (PC). PC is initially dependent on androgens, and advanced PC is treated with androgen deprivation therapy (ADT). When PC progresses despite ADT, they are deemed castration resistant (CRPC). Although CRPC can effectively be treated with potent AR targeted therapies such as abiraterone and enzalutamide, it eventually becomes refractory to such treatments. The glucocorticoid receptor (GR) signaling contributes to CRPC progression. GR is a similar hormone receptor to AR that shares transcriptional targets and may contribute to CRPC progression. Overexpression and/or activation of GR can compensate for AR blockade and lead to enzalutamide-resistant (Enza-R) cancer cell survival. Selective glucocorticoid receptor modulators (SGRMs) decrease GR's activity and delay CRPC growth in preclinical PC models. BET-family proteins can facilitate AR and GR signaling, and BET bromodomainproteins interact with GR to facilitate transcription. It is contemplated that ABT-075 will diminish Enza-R CRPC progression through dual disruption of both GR and AR transcriptional activity,

The inventors sought to identify DNA enhancer elements through which GR and BRD4 cooperate to enable sustained cell survival signaling subsequent to AR blockade with enzalutamide. It is contemplated that a BETi, such as ABT-075, can reverse GR activation in CPRC cell lines. The inventors will test when GR regulated genes are facilitated by BRD's when AR is blocked and whether GR and BRD4 localize to same enhancer regions upstream of GR-regulated survival/proliferation genes?

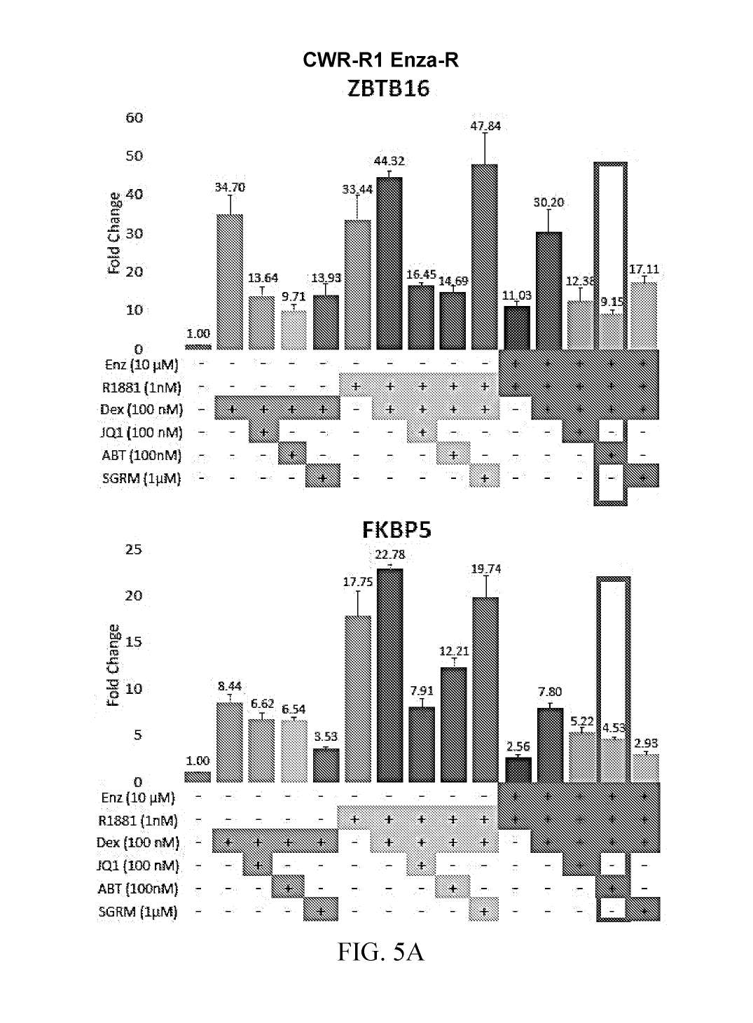

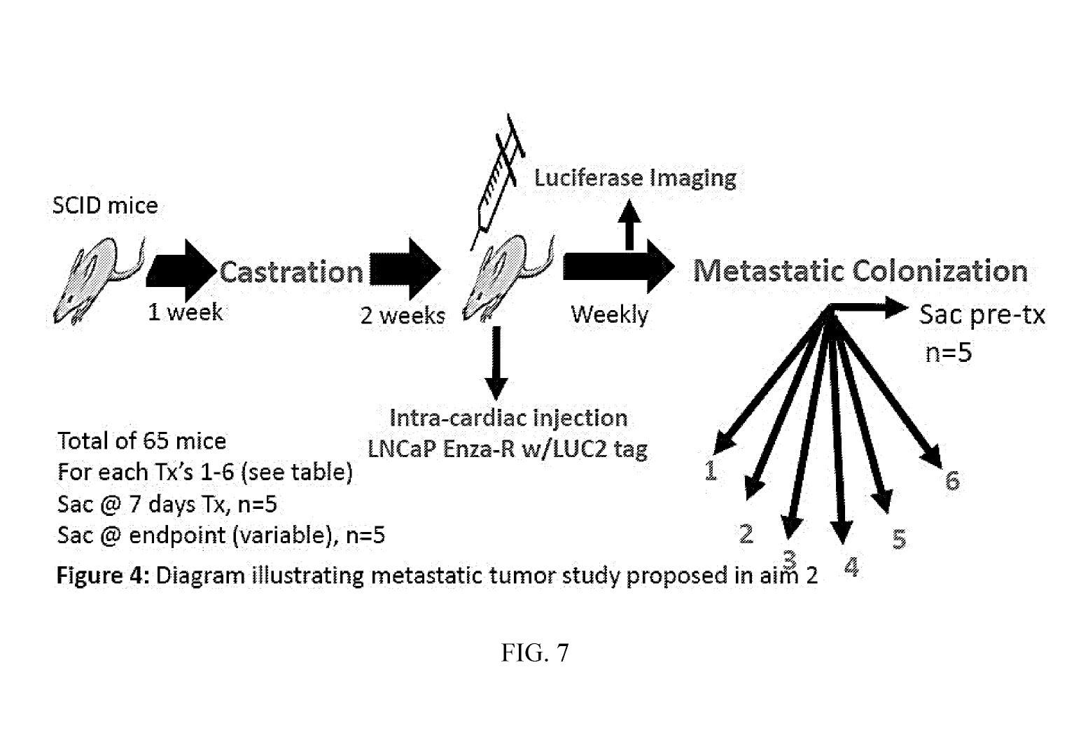

The inventors also sought to determine whether ABT-075 prolongs metastatic GR(+) Enza-R CRPC survival in vivo. This can be tested using immunohistochemistry in a metastatic tumor study (FIG. 7). It was also sought to determine wither BET inhibition provides thapeutic benefit beyond AR/GR blockade and whether BET inhibition is sufficient to provide AR and GR inhibition. The cell lines used in these experiments include two genetically distinct PC cell lines, grown in vitro for over three months in enzalutamide. The PC cell lines become Enza-R with high GR expression, CRPC metastatic in vivo. The LNCaPEnza-R is AR with ligand binding domain mutation (T878A). The CWR-R1 Enza-R is AR with ligand binding (H875Y)+AR splice mutations. The effect of ABT=075 on AR/GF, BRD3/4 expression in Enza-R CPRC cell lines is shown in FIG. 4.

Shown in the table below is the GR target genes of interest and their respective GR binding peak distances from the transcription start site (bp) in CWR-22Rv1 cells treated with R1881, enzalutamide and dexamethasone.

| Gene | Proximal | Distal | |
|---|---|---|---|
| Name | (>10 kbp) | (10-100 kbp) | Distance from TSS (bp) |
| FKBP5 | + | − | −3604, −3304, −3004, 0, 297, 597, |
| | | | 1197, 1497, 1797, 2097, 2697, |
| | | | 2397, 2997, 5397, 5697, 5997, |
| | | | 6297 |
| ZBTB16 | − | + | −22831, 50413, 53713, 54013, |
| | | | 61213 |

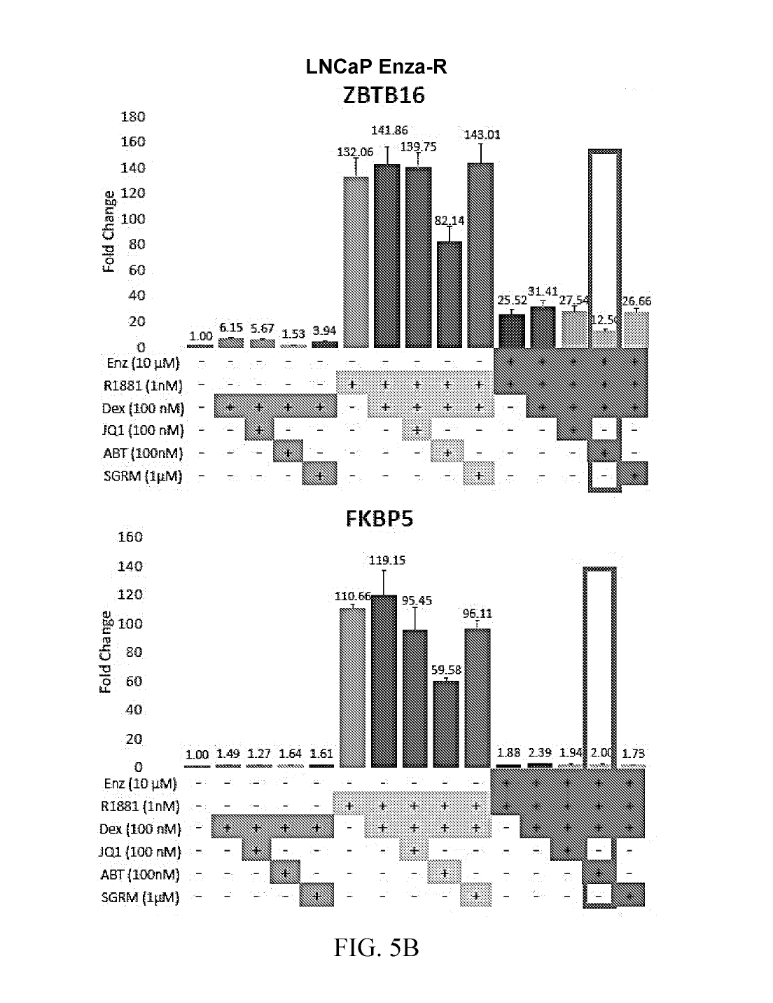

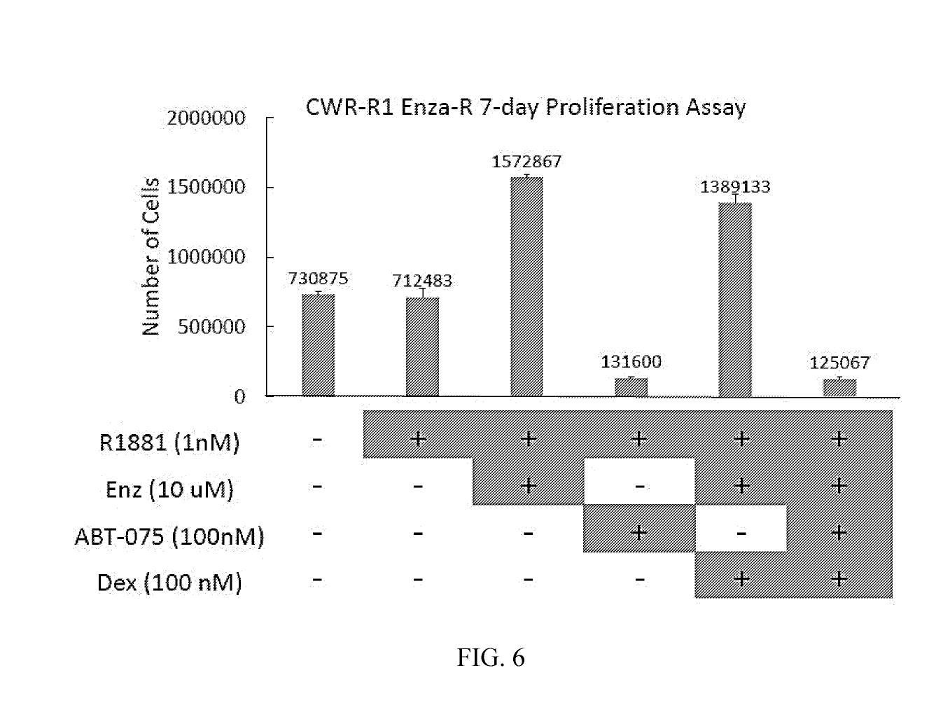

As shown in FIG. 5A-B, it was found that ABT-075 blocks GR-mediated transcriptional activity, and FIG. 6 demonstrates that BET inhibition delays CRPC proliferation. In conclusion, it was found that BRD3, BRD4 are expressed in models of Enza-R CRPC, BETi with ABT-075 can affect AR/GR/BRD expression, ABT-075 represses GR transcriptional activity in human prostate cancer cell lines (most notably for ZBTB16 which has a distal GR binding peak), and ABT-075 (100 nM) potently diminishes Enza-R cell survival in vitro.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

1. A method for treating androgen receptor positive (AR+) and/or glucocorticoid receptor positive (GR+) prostate cancer in a patient comprising administering an effective amount of a BET inhibitor in combination with one or both of an anti-androgen and a glucocorticoid receptor (GR) modulator.

2. A method of inhibiting proliferation of glucocorticoid receptor positive (GR+) and/or androgen receptor (AR+) breast or prostate cancer cells comprising administering to the cells an effective amount of a BET inhibitor in combination with one or both of a chemotherapeutic agent and an anti-androgen

3. The method of claim 1 or 2, wherein the cancer is AR+ prostate cancer.

4. The method of claim 3, wherein the cancer is GR+ prostate cancer.

5. The method of claim 1 or 2, wherein the cancer is castration resistant proste cancer.

6. The method of claim 5, wherein the patient has been determined to be resistant to an anti-androgen therapy.

7. The method of claim 6, wherein the patient has beed determined to be resistant to enzalutamide.

8. The method of claim 1 or 2, wherein the method comprises the administration of a BET inhibitor in combination with an anti-androgen.

9. The method of claim 1 or 2, wherein the method comprises administration of a BET inhibitor in combination with a GR modulator.

10. The method of claim 9, wherein the method further comprises administration of an anti-androgen.

11. A method of inhibiting proliferation of glucocorticoid receptor positive (GR+) and/or androgen receptor (AR+) breast or prostate cancer cells comprising administering to the cells an effective amount of a BET inhibitor in combination with one or both of a chemotherapeutic agent and a glucocorticoid receptor modulator.

12. A method for treating a GR+ and/or AR+ breast or prostate cancer in a patient comprising administering an effective amount of a BET inhibitor in combination with one or both of a chemotherapeutic agent and a glucocorticoid receptor modulator.

13. The method of claim 11 or 12, wherein the wherein the cells are breast cancer cells or the cancer is breast cancer.

14. The method of claim 13, wherein the breast cancer is triple negative breast cancer (TNBC).

15. The method of claim 11 or 12, wherein the cells are prostate cancer cells or the cancer is prostate cancer.

16. The method of claim 15, wherein the cancer is castration resistant prostate cancer or the cells are a castratation resistant prostate cancer cell line.

17. The method of any one of claims 11-16, wherein the cells or cancer are AR+.

18. The method of any one of claims 11-17, wherein the cells or cancer are GR+.

19. The method of any one of claims 12-18, wherein the patient has been determined to have cancer cells that are AR+.

20. The method of any one of claims 15-19, wherein the method comprises administration of a BET inhibitor and one or more chemotherapeutic agent, wherein the chemotherapeutic agent comprises one or more of docetaxel, cabazitaxel, mitoxantrone, abiraterone, prednisone, radium-223, sipuleucel-T, mitoxantrone, bicalutamide, flutamide, nilutamide, ketoconazole, and low-dose corticosteroids.

21. The method of any one of claims 15-20, wherein the method further comprises administration of an antiandrogen.

22. The method of claim 21, wherein the antiandrogen comprises enzalutamide.

23. The method of any one of claims 15-22, wherein the cells or cancer are chemo-resistant.

24. The method of any one of claims 15-23, wherein the cells or cancer are resistant to antiandrogens.

25. The method of claim 24, wherein the cells or cancer are resistant to enzalutamide.

26. The method of any one of claims 15-25, wherein the patient has been determined to have enzalutamide-resistant prostate cancer.

27. The method of any one of claims 12-26, wherein the patient has previously been treated for breast or prostate cancer.

28. The method of claim 27, wherein the patient has previously been treated with one or more chemotherapeutic agents.

29. The method of claim 27 or 28, wherein the patient has been determined to be chemo-resistant or have a reduced sensitivity to a chemotherapeutic agent.

30. The method of any one of claims 12-29, wherein the patient is determined to have cancer cells that are GR+

31. The method of any one of claims 11-30, wherein the patient is determined to have breast cancer cells that are PR negative, ER negative, and HER-2 negative.

32. The method of any one of claims 11-31, wherein the patient is one that has been diagnosed as having GR+ cancer.

33. The method of any one of claims 11-32, wherein the patient is one that has been diagnosed as having TNBC.

34. The method of any one of claims 11-33, wherein the BET inhibitor and the glucocorticoid receptor modulator and/or chemotherapeutic agent are administered within one week of each other.

35. The method of claim 34, wherein the combination of anti-cancer compounds is administered within 24 hours of each anti-cancer compound.

36. The method of claim 34, wherein the BET inhibitor is administered prior to or after the glucocorticoid receptor modulator.

37. The method of claim 34 wherein the BET inhibitor is administered prior to or after the chemotherapeutic agent.

38. The method of any one of claims 11-37, wherein the method comprises the administration of a GR modulator.

39. The method of claim 38, wherein the glucocorticoid receptor modulator comprises RU-486, RU-43044, RU-38486, CP-409069 ORG 214007, ORD ZK-216348, CORT 125134, GSK 9027, AL-438, ZK 245186, CmdA, BI115, Quinol-4-ones, LGD5552, ZK 216348, or analogs or metabolites thereof.

40. The method of any one of claims 11-39, wherein the BET inhibitor comprises JQ1, I-BET 151, I-BET 762, OTX-015, TEN-010, CPI-203, RVX-208, LY294002, MK-8628, BMS-986158, INCB54329, ABBV-075, CPI-0610, FT-1101, GS-5829, and PLX51107.

41. The method of any one of claims 11-40, wherein the method comprises administration of one or more chemotherapeutic agents.

42. The method of claim 41, wherein the chemotherapeutic agent comprises one or more of capecitabine, carboplatin, cyclophosphamide, daunorubicin, docetaxel, doxorubicin, epirubicin, fluorouracil, gemcitabine, eribulin, ixabepilone, methotrexate, mitomycin C, mitoxantrone, paclitaxel, thiotepa, vincristine, or vinorelbine.

43. The method of any one of claims 11-42, wherein the method further comprises categorizing the patient as ER+ or ER− based the level of estrogen receptor expression and a predetermined threshold value for ER expression.

44. The method of any one of claims 11-43, wherein the method further comprises categorizing the patient as GR+ or GR− based the level of glucocorticoid receptor expression and a predetermined threshold value for GR expression.

45. The method of any one of claims 11-44, wherein the method further comprises categorizing the patient as PR+ or PR− based the level of progesterone expression and a predetermined threshold value for PR expression.

46. The method of any one of claims 11-45, wherein the method further comprises categorizing the patient as HER-2+ or HER-2-negative-based the level of HER-2 expression and a predetermined threshold value for HER-2 expression.

47. The method of any one of claims 11-46, wherein the method further comprises categorizing the patient as AR+ or AR-negative-based on the level of AR expression and a predetermined threshold value for AR expression.

48. The method of any one of claims 43-47, wherein the predetermined threshold value identifies a patient as positive if the patient's expression level is in the 25^{th} percentile or greater compared to a normalized sample.

49. The method of claim 48, wherein the normalized sample is based on one or more cancer samples.

50. The method of any one of claims 43-49, wherein the predetermined threshold value for GR activity is dependent on whether the patient is categorized as ER+ or ER−.

51. The method of claim 50, wherein the predetermined threshold value for GR activity identifies a patient as GR+ if the patient is ER− and GR activity level is in the 65^{th} percentile or greater compared to a normalized sample.

52. The method of claim 51, wherein the normalized sample is based on one or more cancer samples.

53. The method of claim 51 or 52, wherein the activity level of GR is assayed by measuring the level of GR expression.

54. The method of claim 53, wherein GR expression is GR transcript expression.

55. The method of claim 53, wherein GR expression is GR protein expression.

56. The method of claim 51 or 52, wherein the activity level of GR is measured by assaying the expression level of one or more GR-responsive genes.

57. The method of claim 56, wherein the GR responsive gene is MCL1, SAP30, DUSP1, SGK1, SMARCA2, PTGDS, TNFRSF9, SFN, LAPTMS, GPSM2, SORT1, DPT, NRP1, ACSLS, BIRC3, NNMT, IGFBP6, PLXNC1, SLC46A3, C14orf139, PIAS1, IDH2, SERPINF1, ERBB2, PECAM1, LBH, ST3GAL5, IL1R1, BIN1, WIPF1, TFPI, FN1, FAM134A, NRIP1, RAC2, SPP1, PHF15, BTN3A2, SESN1, MAP3K5, DPYSL2, SEMA4D, STOM, MAOA, AKAP1, AREG, ARHGEF26, BIRC3, CA12, CALCR, CDC42EP3, CYP24A1, DEPTOR, DOCK4, DUSP6, FGF18, FOS, GAD1, GREB1, IL6R, IL6ST, KAZN, KCNJ8, KDM4B, KIAA0226L, KLF9, LAMA3, MAFB, MYC, NR5A2, PERI, PHLDA1, PSCA, RGS2, RHOBTB1, SGK1, SNAI2, SOCS2, SYBU, TBC1D8, TGF A, WIPF1, WWC1, ALDH1A3, CXCL12, LRRC15, LY6H, NR4A2, PDZK1, PPIF. SLC22A4, RNF43, ARL14, CD44, CYP1A1, DDX10, EGR3, EMP1, FJX1, HCK, HEG1, HEY2, PTGES, RAB31, RARA, SIM1, SLC26A2, TMEM120B, TNFRSF11B, TRPC6, DIRAS2, KRT13, LRP4, PTGER4, RET, RGCC, SEMA3B, SERPINB9, SLC47A1, SUV39H2, RAPGEFL1, MICB, HS3ST3A1, HSPB8, IGFBP4, JAK2, KIT, LEF1, LINC00341, MAFF MYBL1, NPY1R, NPY5R, PGR, PLAC1, or PMAIP1.

58. A method for treating a triple-negative breast cancer patient determined to be GR+ comprising administering a BET inhibitor and administering a chemotherapeutic agent and/or a glucocorticoid receptor modulator.

59. The method of claim 58, wherein the patient was previously determined to be chemotherapy-resistant.

60. A method of inhibiting proliferation of prostate cancer cells comprising administering to the cells an effective amount of a BET inhibitor in combination with one or both of an anti-androgen and a glucocorticoid receptor modulator.

61. A method for treating prostate cancer in a patient comprising administering an effective amount of a BET inhibitor in combination with one or both of an anti-androgen and a glucocorticoid receptor modulator.

62. The method of claim 60 or 61, wherein the wherein the cells or cancer are GR+.

63. The method of any one of claims 60-62, wherein the cancer is castration resistant prostate cancer or the cells are a castratation resistant prostate cancer cells.

64. The method of any one of claims 60-63, wherein the cells or cancer are AR+.

65. The method of any one of claims 61-63, wherein the patient has been determined to have cancer cells that are AR+.

66. The method of any one of claims 60-65, wherein the method further comprises administration of a chemotherapeutic agent.

67. The method of claim 66, wherein the chemotherapeutic agent comprieses one or more of docetaxel, cabazitaxel, mitoxantrone, abiraterone, prednisone, radium-223, sipuleucel-T, mitoxantrone, bicalutamide, flutamide, nilutamide, ketoconazole, and low-dose corticosteroids.

68. The method of any one of claims 60-67, whereinn the antiandrogen comprises enzalutamide.

69. The method of any one of claims 60-68, wherein the cells or cancer are chemo-resistant.

70. The method of any one of claims 60-69, wherein the cells or cancer are resistant to antiandrogens.

71. The method of claim 70, wherein the cells or cancer are resistant to enzalutamide.

72. The method of any one of claims 60-71, wherein the patient has been determined to have enzalutamide-resistant prostate cancer.

73. The method of any one of claims 61-72, wherein the patient has previously been treated for prostate cancer.

74. The method of claim 73, wherein the patient has previously been treated with one or more anti-androgens or one or more chemotherapeutic agents.

75. The method of claim 73 or 74, wherein the patient has been determined to be chemo-resistant, resistant to the anti-androgen, or have a reduced sensitivity to a chemotherapeutic agent or an anti-androgen.

76. The method of any one of claims 61-75, wherein the patient is determined to have or diagnosed as having cancer cells that are GR+

77. The method of any one of claims 60-76, wherein the BET inhibitor and the anti-androgen are administered within one week of each other.

78. The method of claim 77, wherein the combination of anti-cancer compounds is administered within 24 hours of each anti-cancer compound.

79. The method of claim 77, wherein the BET inhibitor is administered prior to or after the anti-androgen.

80. The method of any one of claims 60-79, wherein the method comprises the administration of a GR modulator.

81. The method of claim 80, wherein the glucocorticoid receptor modulator comprises RU-486, RU-43044, RU-38486, CP-409069 ORG 214007, ORD ZK-216348, CORT 125134, GSK 9027, AL-438, ZK 245186, CmdA, BI115, Quinol-4-ones, LGD5552, ZK 216348, or analogs or metabolites thereof.

82. The method of any one of claims 60-81, wherein the BET inhibitor comprises JQ1, I-BET 151, I-BET 762, OTX-015, TEN-010, CPI-203, RVX-208, LY294002, MK-8628, BMS-986158, INCB54329, ABBV-075, CPI-0610, FT-1101, GS-5829, and PLX51107.

83. The method of any one of claims 60-82, wherein the method further comprises categorizing the patient as GR+ or GR− based the level of glucocorticoid receptor expression and a predetermined threshold value for GR expression.

84. The method of any one of claims 60-83, wherein the method further comprises categorizing the patient as PR+ or PR− based the level of progesterone expression and a predetermined threshold value for PR expression.

85. The method of any one of claims 60-84, wherein the method further comprises categorizing the patient as AR+ or AR-negative-based on the level of AR expression and a predetermined threshold value for AR expression.

86. The method of any one of claims 83-85, wherein the predetermined threshold value identifies a patient as positive if the patient's expression level is in the 25^{th} percentile or greater compared to a normalized sample.

87. The method of claim 86, wherein the normalized sample is based on one or more cancer samples.

88. The method of any one of claims 60-87, wherein the method further comprises determining the activity or expression level of GR in a biological sample from the patient.

89. The method of claim 88, wherein the activity level of GR is assayed by measuring the level of GR expression.

90. The method of claim 89, wherein GR expression is GR transcript expression.

91. The method of claim 89, wherein GR expression is GR protein expression.

92. The method of claim 89, wherein the activity level of GR is measured by assaying the expression level of one or more GR-responsive genes.

93. The method of claim 92, wherein the GR responsive gene is MCL1, SAP30, DUSP1, SGK1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, IDH2, SERPINF1, ERBB2, PECAM1, LBH, ST3GAL5, IL1R1, BIN1, WIPF1, TFPI, FN1, FAM134A, NRIP1, RAC2, SPP1, PHF15, BTN3A2, SESN1, MAP3K5, DPYSL2, SEMA4D, STOM, MAOA, AKAP1, AREG, ARHGEF26, BIRC3, CA12, CALCR, CDC42EP3, CYP24A1, DEPTOR, DOCK4, DUSP6, FGF18, FOS, GAD1, GREB1, IL6R, IL6ST, KAZN, KCNJ8, KDM4B, KIAA0226L, KLF9, LAMA3, MAFB, MYC, NR5A2, PERI, PHLDA1, PSCA, RGS2, RHOBTB1, SGK1, SNAI2, SOCS2, SYBU, TBC1D8, TGFA, WIPF1, WWC1, ALDH1A3, CXCL12, LRRC15, LY6H, NR4A2, PDZK1, PPIF. SLC22A4, RNF43, ARL14, CD44, CYP1A1, DDX10, EGR3, EMP1, FJX1, HCK, HEG1, HEY2, PTGES, RAB31, RARA, SIM1, SLC26A2, TMEM120B, TNFRSF11B, TRPC6, DIRAS2, KRT13, LRP4, PTGER4, RET, RGCC, SEMA3B, SERPINB9, SLC47A1, SUV39H2, RAPGEFL1, MICB, HS3ST3A1, HSPB8, IGFBP4, JAK2, KIT, LEF1, LINC00341, MAFF MYBL1, NPY1R, NPY5R, PGR, PLAC1, or PMAIP1.
